(19) United States
(12) Patent Application Publication
(10) Pub. No.: US 2008/0213812 A1
(43) Pub. Date: Sep. 4, 2008

(54) Methods and compositions for modulating telomerase reverse transcriptase (TERT) expression

(76) Inventors: William H. Andrews, Reno, NV (US); Laura Briggs, Reno, NV (US); Christopher Foster, Reno, NV (US); Hamid Mohammadpour, Reno, NV (US); Stephanie Fraser, Sparks, NV (US); David Monda, Sparks, NV (US); Lancer Brown, Sparks, NV (US); Frederick M. Hahn, San Rafael, CA (US); Ronald Pruzan, Palo Alto, CA (US); David Schooley, Reno, NV (US); Jason March, Reno, NV (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/009,623

(22) Filed: Jan. 18, 2008

Related U.S. Application Data
(63) Continuation of application No. 11/088,001, filed on Mar. 22, 2005, now abandoned.
Application No. 11/088,001 is a continuation-in-part of application No. 10/951,906, filed on Sep. 29, 2004, now abandoned.
(60) Provisional application No. 60/557,949, filed on Mar. 30, 2004.
(60) Provisional application No. 60/507,271, filed on Sep. 29, 2003.

(51) Int. Cl. C12Q 1/48; C12N 5/06

(52) U.S. Cl. 435/15; 435/375

(57) ABSTRACT

Methods and compositions are provided for modulating, e.g., increasing or decreasing, the expression of telomerase reverse transcriptase (TERT). In the subject methods, the binding interaction of the TERT Site C repressor site with a Site C repressor protein complex made up of one or more proteins is modulated to achieve the desired change in TERT expression. A feature of the subject invention is that the target Site C repressor protein complex includes an LSF protein. The subject methods and compositions find use in a variety of different applications, including the immortalization of cells, the production of reagents for use in life science research, therapeutic applications; therapeutic agent screening applications; and the like.

### CROSS-REFERENCE TO RELATED APPLICATIONS

This application is a continuation in part of application Ser. No. 10/951,906 filed on Sep. 29, 2004; which application, pursuant to 35 U.S.C. §119 (e), claims priority to the filing date of U.S. Provisional Patent Application Ser. No. 60/557,949 filed Mar. 30, 2004 and to the filing date of U.S. Provisional Patent Application Ser. No. 60/507,271 filed on Sep. 29, 2003; the disclosures of which applications are herein incorporated by reference.

### INTRODUCTION

1. Background of the Invention

Telomeres, which define the ends of chromosomes, consist of short, tandemly repeated DNA sequences loosely conserved in eukaryotes. For example, human telomeres consist of many kilobases of (TTAGGG)n together with various associated proteins. Small amounts of these terminal sequences or telomeric DNA are lost from the tips of the chromosomes during S phase because of incomplete DNA replication. Many human cells progressively lose terminal sequence with cell division, a loss that correlates with the apparent absence of telomerase in these cells. The resulting telomeric shortening has been demonstrated to limit cellular lifespan.

Telomerase is a ribonucleoprotein that synthesizes telomeric DNA. In general, telomerase is made up of two components: (1) an essential structural RNA (TR or TER) (where the human component is referred to in the art as hTR or hTER); and (2) a catalytic protein (telomerase reverse transcriptase or TERT) (where the human component is referred to in the art as hTERT). Telomerase works by recognizing the 3′ end of DNA, e.g., telomeres, and adding multiple telomeric repeats to its 3′ end with the catalytic protein component, e.g., hTERT, which has polymerase activity, and hTER which serves as the template for nucleotide incorporation. Of these two components of the telomerase enzyme, both the catalytic protein component and the RNA template component are activity-limiting components.

Because of its role in cellular senescence and immortalization, there is much interest in the development of protocols and compositions for regulating telomerase activity.

2. Relevant Literature

WO 03/016474; WO 03/000916; WO 02/101010; WO 02/090571; WO 02/090570; WO 02/072787; WO 02/070668; WO 02/16658; WO 02/16657 and the references cited therein.

### SUMMARY OF THE INVENTION

Methods and compositions are provided for modulating, e.g., increasing or decreasing, the expression of telomerase reverse transcriptase (TERT), in a cell. In the subject methods, the cell is contacted with a TERT promoter regulator in a manner sufficient to modulate TERT expression. The subject methods and compositions find use in a variety of different applications.

### BRIEF DESCRIPTION OF THE FIGURES

FIG. 1 shows hTERT promoter including Site C and GC-Box contained therein.

FIG. 2 shows the LSF matrix table.

FIG. 3 shows alignment of LBP1c and LBP1c2.

FIG. 4 shows alignment of BOM and BOMv2.

FIG. 5 shows reported LSF sites in various promoters.

FIG. 6 shows representative antibodies for LSF family members.

FIG. 7 shows representative Site C decoy sequences

FIG. 8 shows dominant negative mutants of Site C proteins.

FIG. 9 shows the general scheme of purification of Site C proteins.

FIG. 10 shows alignment of Site C and OBC.

### DESCRIPTION OF THE SPECIFIC EMBODIMENTS

Methods and compositions are provided for modulating, e.g., increasing or decreasing, the expression of telomerase reverse transcriptase (TERT), in a cell. In the subject methods, the cell is contacted with a TERT promoter regulator in a manner sufficient modulate TERT expression. The subject methods and compositions find use in a variety of different applications.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Methods recited herein may be carried out in any order of the recited events which is logically possible, as well as the recited order of events.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

In further describing the subject invention, the methods and compositions of the invention are described first in greater detail, followed by a review of the various applications in which the subject invention finds use.

### Methods

Aspects of the invention include modulating the activity of TERT, e.g., in a cell, by contacting the cell with a TERT promoter regulator, which modulates the activity of TERT promoter thereby modulating the activity of TERT, e.g., the expression of TERT, including the transcription, translation, or steady state level of TERT or activation or any functional activity of TERT. The term “modulating” is used to refer to either “increasing” or “decreasing” expression of TERT. As such, in certain embodiments, methods of increasing expression of TERT are provided, while in other embodiments, methods of decreasing expression of TERT are provided. As developed in more detail below, the TERT promoter regulator of the present invention can be any agent or entity that regulates the promoter activity of TERT. For example, the TERT promoter regulator can be any agent or entity that regulates the activity of one or more protein binding regions of TERT promoter.

In one embodiment, the TERT promoter regulator is a Site C regulator. The Site C regulator of these embodiments includes any agent or entity capable of regulating the activity of one or more repressor sites in a TERT promoter. Put another way, TERT expression is modulated by modulating the TERT expression repression activity of a Site C repressor binding site located in the TERT minimal promoter, where modulating includes both increasing and decreasing the expression repressive activity of the Site C repressor binding site.

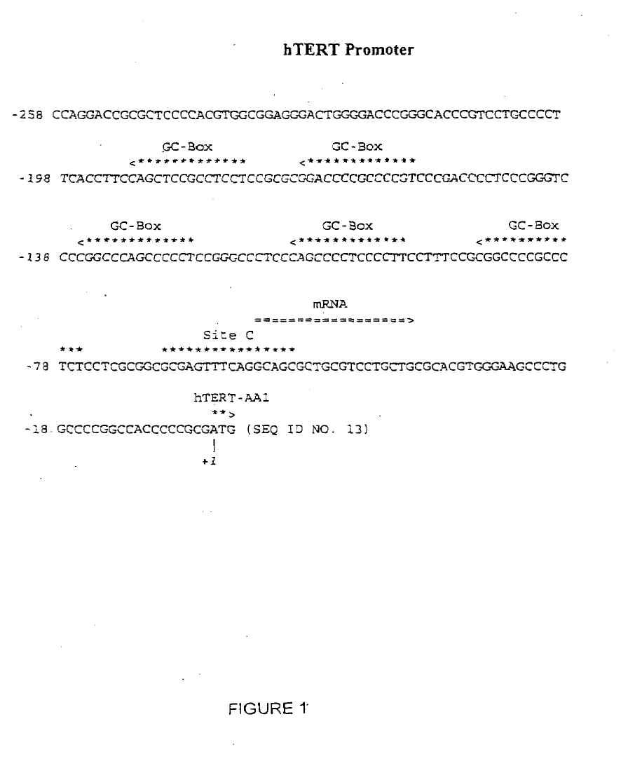

In the human TERT minimal promoter, there are one or more repressor sites within the region of 1 to −100, −40 to −90, −50 to −90, or −50 to −70 (with the position relative to the “A” of ATG start codon for TERT). In particular, there are one or more repressor sites within the region of −89 to 48 of hTERT promoter including, without any limitation, Site C within the region of −66 to −51 and GC-Box within the region of −89 to −76 as shown in FIG. 1. Repressor sites in TERT promoter have also been described in the U.S. Pat. No. 6,686,159, which is incorporated herein by reference. In certain embodiments, the Site C sequence is:

(SEQ ID NO:33)

GGCCCCGCCCTCTCCTCGCGGCGCGAGTTTCAGGCAGCGCT

In certain embodiments, the target Site C sequence is a portion or subsequence of the above sequence, such as:

| | |
|---|---|
| GGCGCGAGTTTCA; | (SEQ ID NO:34) |
| CGCGAGTTTC; | (SEQ ID NO:35) |
| or | |
| GGCGCGAGTTTCAGGCAGCGC. | (SEQ ID NO:36) |

Also of interest are Site C sites that have a sequence that is substantially the same as, or identical to, the Site C repressor binding site sequences as described above, e.g., SEQ ID NOs: 34-37. A given sequence is considered to be substantially similar to this particular sequence if it shares high sequence similarity with the above described specific sequences, e.g., at least 75% sequence identity, usually at least 90%, more usually at least 95% sequence identity with the above specific sequences. Sequence similarity is calculated based on a reference sequence, which may be a subset of a larger sequence. A reference sequence will usually be at least about 10 nt long, more usually at least about 12 nt long, and may extend to the complete sequence that is being compared. Algorithms for sequence analysis are known in the art, such as BLAST, described in Altschul et al. (1990), J. Mol. Biol. 215:403-10 (using default settings, i.e. parameters w=4 and T=17). Of particular interest in certain embodiments are nucleic acids of substantially the same length as the specific nucleic acid identified above, where by substantially the same length is meant that any difference in length does not exceed about 20 number %, usually does not exceed about 10 number % and more usually does not exceed about 5 number %; and have sequence identity to this sequence of at least about 90%, usually at least about 95% and more usually at least about 99% over the entire length of the nucleic acid. Also of interest are nucleic acids that represent a modified or altered Site C site, e.g., where the site includes one or more deletions or substitutions as compared to the above specific Site C sequences, including a deletion or substitution of a portion of the Site C repressor binding site, e.g., a deletion or substitution of at least one nucleotide.

As summarized above, aspects of the invention include contacting a cell with a TERT promoter, and more specifically a Site C, regulator in a manner sufficient to achieve the desired modulation of TERT expression. According to the present invention, agents or entities capable of regulating the activity of a repressor site in a TERT promoter include agents that interact directly or indirectly with the repressor site, e.g., thereby changing the repressor site's function or activity level with respect to the promoter activity or the activity of TERT. For example, a Site C regulator includes an agent or entity capable of directly binding to or interacting with one or more repressor sites in TERT promoter. Alternatively a Site C regulator includes an agent or entity capable of binding to, interacting with, or modulating the activity of one or more agents that directly bind to or interact with one or more repressor sites in a TERT promoter.

Any convenient Site C regulator may be employed in the subject methods, where representative Site C regulators are reviewed below. Use of a given regulator in a given application will depend on certain factors, e.g., whether activity of the TERT promoter is to be enhanced or decreased, whether the application is in vitro or in vivo, etc. In general, a Site C regulator of the present invention can be any suitable agent or entity, including without any limitation, polypeptide, protein, factor, ligand, antibody, peptide, peptide aptamer, chemical compound, polynucleotide, oligonuleotide, double-stranded oligonucleotide, double-stranded RNA, e.g. RNAi, antisense, ribozyme, aptamer, etc.

In one embodiment, the Site C regulator of the present invention is a Site C protein. The Site C protein of the present invention includes a polypeptide, protein, protein fragment, or peptide capable of modulating the activity of one or more repressor sites associated with Site C in TERT promoter. For example, the Site C protein of the present invention can be a protein or a fragment thereof, e.g., DNA binding region or domain that specifically binds to or interacts-with one or more repressor sites associated with Site C in TERT promoter. Examples of such Site C proteins include, without any limitation, HKR3, ZNF140, ZFP161, Solute Carrier Family 3, Splicing Factor 3A, Ran-GTP, ELG, BCL6, Matrin3, BMAL2, U2 snRNA Protein, LZ16, PC4, F13, E2F3B, E2F3, p107, TCFL5, p65, c-Rel, Proteosome, p42POP, WBSCR2, NF45, CA150, Huntingtin, p231HBP, MRG15, ZNF135, Ras GTPase, and PHD7 and any homologs thereof.

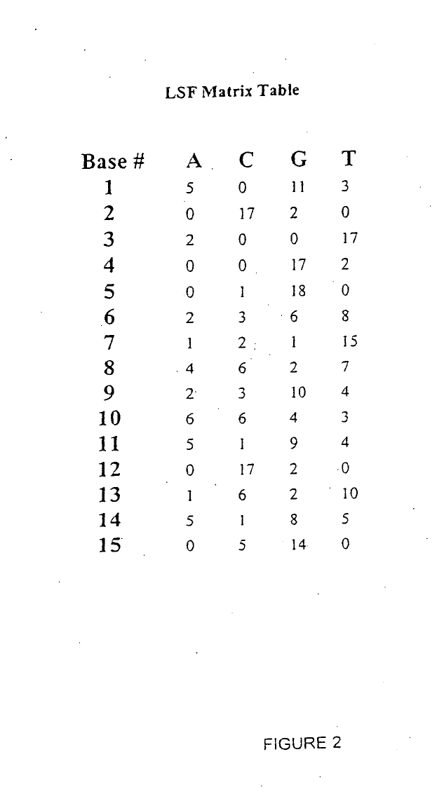

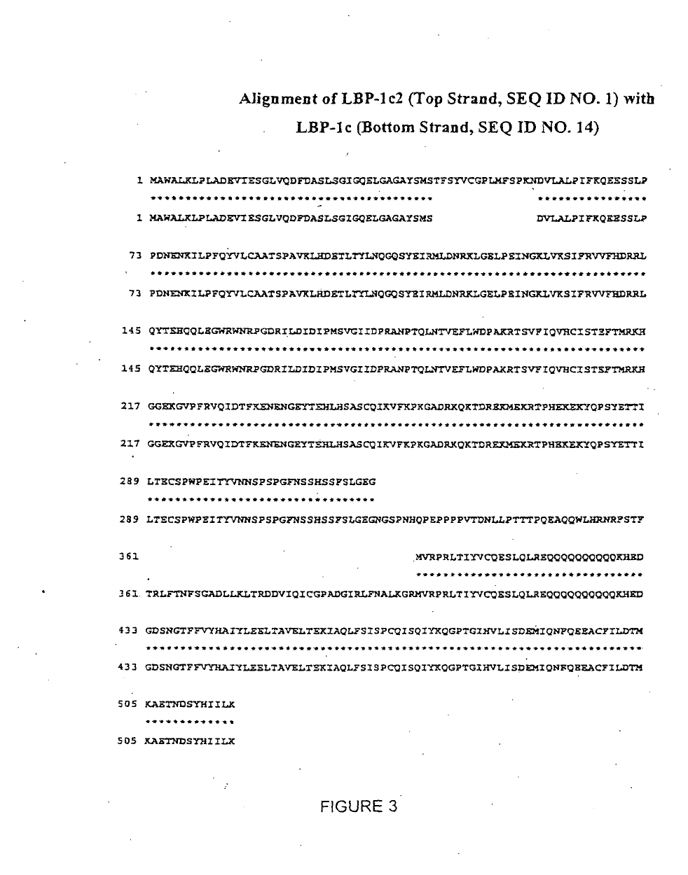

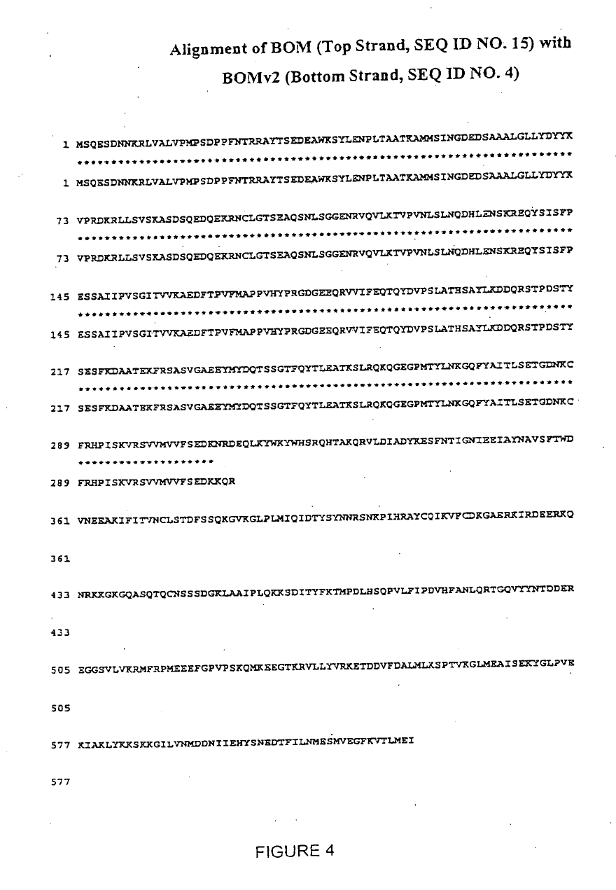

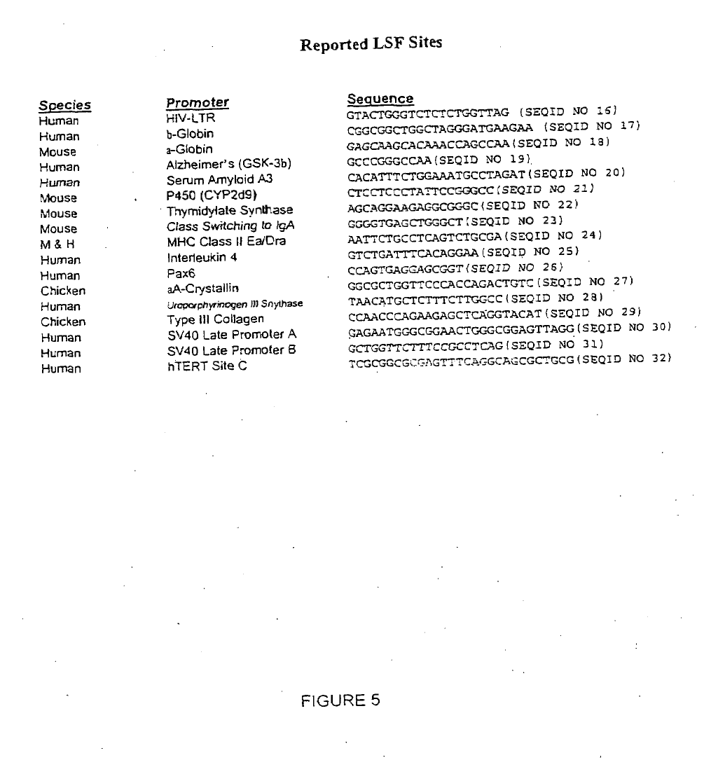

In addition, the Site C protein of the present invention in certain embodiments also includes any polypeptide or protein capable of binding to a DNA region or fragment containing Site C consensus sequence or LSF consensus sequence, e.g., as shown in the LSF matrix table as shown in FIG. 2 and also described in Frith et al., (Frith et al., Bioinformatic 17(10), 878-889 (2001). According to the present invention, examples of the Site C protein also include any known or later discovered members of LSF family including any homolog or any protein or polypeptide with at least 50%, 70%, or 90% of its amino acids identical to a member of LSF family, especially within its functional regions, e.g., its DNA binding domain or regions involved in protein-protein interaction. In general, LSF family is a family of proteins related to mammalian transcription factor LSF. Members of LSF family usually include LBP1a, LBP1b, LBP1c, LBP1d, LBP9, LBP32v1, LBP32v2, SOMv1, SOMv2, SOMv3, and BOM. LBP1d is a splice variant of LBP1c while LBP1a is a splice variant of LBP1b. In addition, the present invention provides LBP1c2 (SEQ ID NOS. 01 and 02), a splice variant of LBP1c and BOMv2 (SEQ ID NOS. 04 and 05), a variant of BOM. As shown in FIG. 3, comparing to LBP1c LBP1c2 has an additional exon (aa 42-56) at the N-terminal region and lacks three consecutive exons (aa 322-399) at the C-terminal region. Similarly as shown in FIG. 4, comparing to BOM, BOMv2 lacks the N-terminal region of BOM, e.g., aa 309 to aa 625 of BOM. According to the present invention, LSF family members also include any protein or polypeptide capable of binding to or interacting with 1) one or more members related to LSF, e.g., YY1, NF-E4, Fe65, APP-CT, NFPB, and SP1 or 2) DNA regions specifically bound by one or more members related to LSF, e.g. LSF sites as shown in FIG. 5.

According to the present invention, the Site C protein of the present invention can also be a GC-Box protein. The GC-Box protein of the present invention includes a polypeptide, protein, protein fragment, or peptide capable of modulating the activity of one or more repressor sites associated with GC-Box in TERT promoter. For example, the GC-Box protein of the present invention can be a protein or a fragment thereof, e.g., DNA binding region or domain that specifically binds to or interacts with one or more repressor sites associated with GC-Box in TERT promoter. Examples of such GC-Box protein include, without any limitation, SP1, SP2, SP3, SP4, SP5, SP6, SP7, SP8, TIEG1, TIEG2, TIEG3, BTEB1, BTEB2, BTEB3, ZF9, ZNF741, UKLF, BKLF, BKLF3, IKLF, GKLF, LKLF, EKLF, KKLF, CPBP, and AP-2rep and any homologs thereof.

In addition, the GC-Box protein of the present invention can also be a polypeptide or protein capable of binding to or interacting with a polypeptide or protein specifically binding to the GC-Box in TERT promoter. Examples of such GC-Box protein include, without any limitation, SP1, TFIIB, TBP, TAF55, TAF135, CRSP, RB, p53, HCF1, KIAA0461, Dorfin, Atf7ip, E2F, Oct1, GATA1, RelA, TIEG, ELF1, SREBP2, Hsc70, SF3A120, HSph2, and KIM1903.

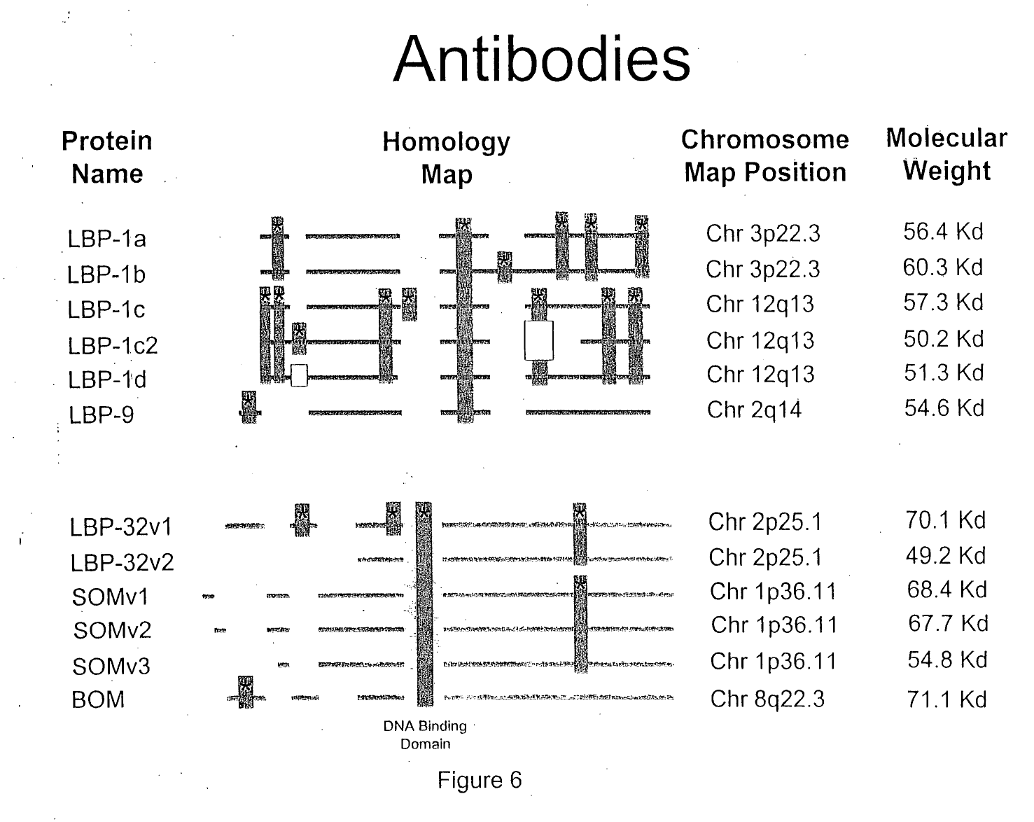

According to another embodiment of the present invention, the Site C regulator of the present invention is a modulator, e.g., inhibitor or activator, of a polypeptide or protein capable of interacting with one or more repressor sites in TERT promoter, e.g., a modulator of Site C proteins including GC-Box proteins and LSF family members. Such modulator includes any agent or entity that regulates the activity of a Site C protein at various levels through any suitable means. For example, such modulator can be an antibody of a Site C protein. As shown in FIG. 6, the present invention provides examples of antibodies specifically binding to epitopes in various regions of members of the LSF family, e.g., DNA binding domain or regions where members of LSF family differ from each other. According to the present invention, such modulators can also be a polypeptide containing the light chain, single chain, variable region, or CDR of the antibody of a Site C protein including humanized antibodies or fragments thereof. Representative antibodies, as well as the representative methods for generating the same, are further described below.

In addition, such modulators can be an aptamer of a Site C protein. Recently, it has been shown that RNA and DNA aptamers can substitute for monoclonal antibodies in various applications (Jayasena, “Aptamers: an emerging class of molecules that rival antibodies in diagnostics.” Clin. Chem., 45(9):1628-50, 1999; Morris et al., “High affinity ligands from in vitro selection: complex targets.” Proc. Natl. Acad. Sci., USA, 95(6):2902-7, 1998).

In general, an aptamer-binds to a non-nucleic acid target molecule and includes an oligonucleotide with a loop portion, a first segment, and a second segment complementary to the first segment, wherein the first and second segments form a stem portion when hybridized together; a binding region formed by the oligonucleotide and configured to bind to the non-nucleic acid target molecule. Additionally these nucleic acid aptamer can also be coupled with various detectable tags or-therapeutic loads, e.g., radioisotope, biotin, fluorescent tags, or toxin. An increasing number of DNA and RNA aptamers that recognize their non-nucleic acid targets has been developed by SELEX and has been characterized (Gold et al., “Diversity of Oligonucleotide Functions,” Annu. Rev. Biochem., 64:763-97, 1995; Bacher & Ellington, “Nucleic Acid Selection as a Tool for Drug Discovery,” Drug Discovery Today, 3(6):265-273, 1998). The relatively fast selection process of the specific aptamers and the inexpensive synthesis makes the aptamer useful alternatives for monoclonal antibodies. These nucleic acids can be easily synthesized, readily manipulated, and can be stored for over long time.

Alternatively the modulator of the present invention includes any agent or entity that regulates Site C protein's interaction with one or more repressor sites associated with Site C in the TERT promoter. For example, such modulator can be a decoy of Site C including one or more repressor sites interacting with a Site C protein. Usually a decoy is a double-stranded, e.g., duplex oligonucleotide which contains one or more transcription factor binding sites capable of binding to and/or competing with the transcription factors that bind to the binding sites within the promoter.

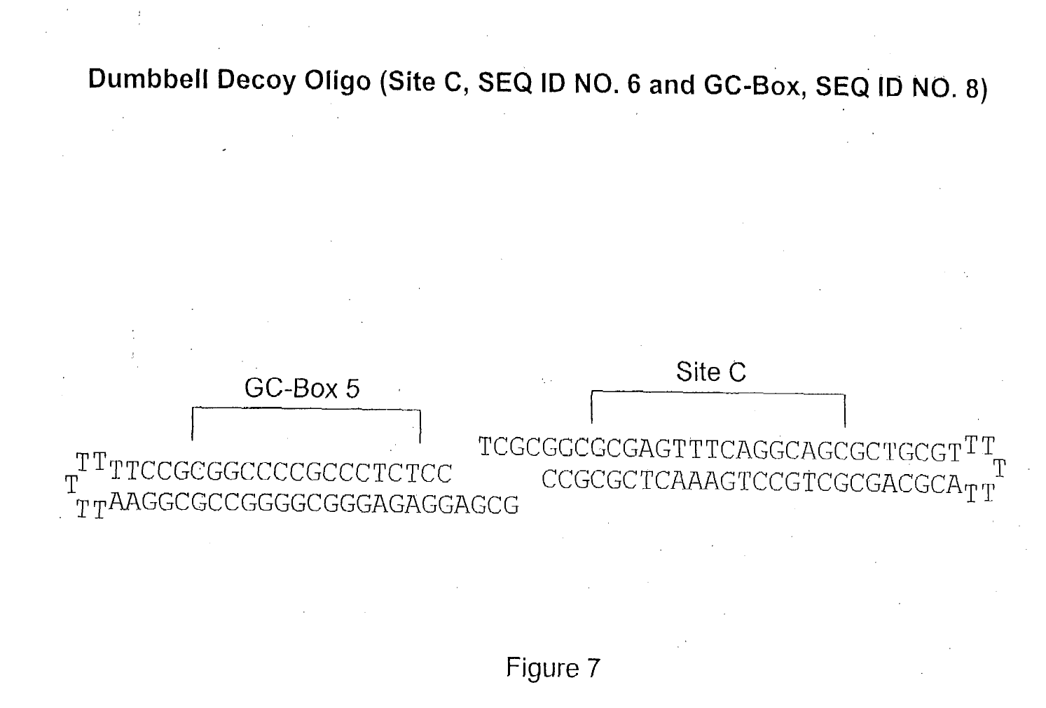

In general, the length of a double-stranded decoy ranges from 10 to 30 nucleotides, e.g., 20 to 30 nucleotides, 30 to 50 nucleotides, or 50 to 100 nucleotides. Oligonucleotide decoys and methods for their use and administration are further described in Morishita et al., Circ Res (1998) 82 (10): 1023-8, which is incorporated herein by reference. Examples of Site C decoys include decoys containing the sequence of Site C, e.g., as shown in SEQ ID NO. 06 (5′-TCGCGGCGCGAGTTTCAGGCAGCGCTGCGTTTTTTACGCAGCGCTGCCTGAAA CTCGCGCC-3′) and SEQ ID NO. 07 (5′-CGGCGCGAGTTTCAGGCAGCGCTG-3′, sequence for one strand) or the sequence of GC-Box, e.g., as shown in SEQ ID NO. 08 (5′-GCGAGGAGAGGGCGGGGCCGCGGAATITIIIICCGCGGCCCCGCCCT-CTCC-3′) and SEQ ID NO. 09 (5′-TCCGCGGCCCCGCCCTCTCCTC-3′, sequence for one strand) or the sequence of both Site C and GC-Box, e.g., as shown in SEQ ID NO. 10 (5′-TCCGCGGCCCCGCCCTCTCCTCGCGGCGCGAGTTTCAGGCAGCGC-TG-3′, sequence for one strand). In general, these sequences form double stranded structures, e.g., a double strand-loop structure as shown in FIG. 7. Alternatively one or more double stranded structures can be ligated to form a bigger structure, e.g., two double strand-loop structures can be ligated together as shown in FIG. 7.

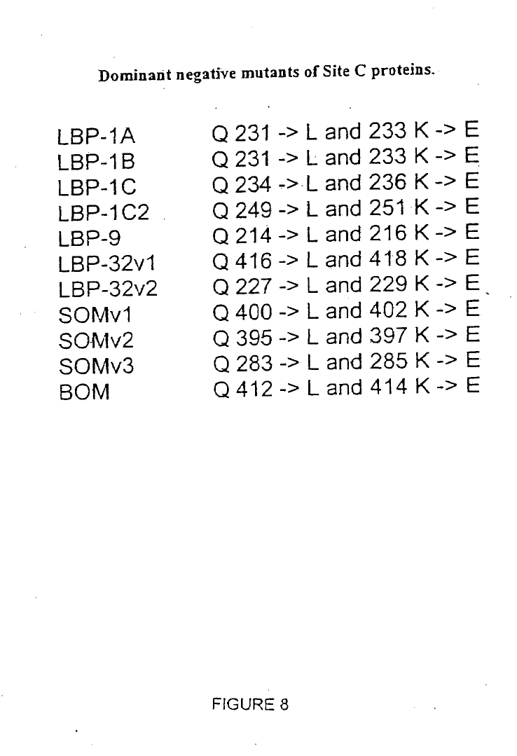

In one embodiment, the Site C regulator of the present invention includes a dominant negative mutant of a Site C protein including GC-Box proteins and LSF family members, e.g., any mutant of a Site C protein lacking the DNA binding domain or having a partially or fully inactivated DNA binding domain. Examples of such mutants for members of LSF family are shown in FIG. 8. In addition, LBP-1d has a mutated DNA binding domain, e.g., an exon containing all or part of the DNA binding domain is deleted via a splicing event, and thus is a dominant negative mutant by itself.

In another embodiment, the Site C regulator of the present invention is a peptide aptamer capable of binding to a Site C regulator, e.g., a Site C protein. In general, a library of random peptides can be expressed as fusions of a detectable protein under the control of a promoter. Such library can be screened for peptides capable of interfering with the activity of a Site C regulator, e.g., blocking the binding of a Site C protein-to one or more repressor sites or reducing the repressing activity of a Site C protein with respect to TERT activity. Methods for screening of peptide aptamer are also described in Blum et al., PNAS (2000) vol. 97 2241-2246, which is incorporated herein by reference.

In yet another embodiment, the Site C regulator of the present invention includes any agent or entity capable of regulating the transcription or translation level of a Site C protein. For example, the promoter of a Site C protein can be targeted directly or indirectly via any means known or later discovered in the field. Promoters of Site C proteins and their regulation are known to one skilled in the art. For example, promoters associated with members of LSF family are described in Swendeman et al., (Swendeman et al., The Journal of Biological Chemistry Vol. 269, No. 15, pp. 11663-11671, 1994). One way to regulate the promoter of a Site C protein gene is to use a decoy of one or more transcription factor binding sites within the promoter of a Site C protein gene.

Alternatively the transcription level of a Site C protein can be regulated by genetically modifying the promoter or gene of a Site C protein, e.g., so that it no longer encodes a functional repressor protein. Genetic modification, alteration or mutation may take a number of different forms, e.g., through deletion, substitution, or addition of one or more nucleotide residues in the promoter or coding region. One means of making such modification is by homologous recombination. Methods for generating targeted gene modifications through homologous recombination are known in the art, including those described in: U.S. Pat. Nos. 6,074,853; 5,998,209; 5,998,144; 5,948,653; 5,925,544; 5,830,698; 5,780,296; 5,776,744; 5,721,367; 5,614,396; 5,612,205; the disclosures of which are herein incorporated by reference.

In addition, the transcription level of a Site C protein can be regulated by gene silencing using double-strand RNA (Sharp (1999) Genes and Development 13: 139-141). RNAi, otherwise known as double-stranded RNA interference (dsRNAi) or small interfering RNA (siRNA), has been extensively documented in the nematode C. elegans (Fire, A., et al, Nature, 391, 806-811, 1998) and routinely used to “knock down” genes in various systems. RNAi agents may be dsRNA or a transcriptional template of the interfering ribonucleic acid which can be used to produce dsRNA in a cell. In these embodiments, the transcriptional template may be a DNA that encodes the interfering ribonucleic acid. Methods and procedures associated with RNAi are also described in WO 03/010180 and WO 01/68836, all of which are incorporated herein by reference.

According to the present invention, the Site C regulator can also be an antisense or ribozyme of a Site C protein. An anti-sense reagent may be antisense oligodeoxynucleotides (ODN), particularly synthetic ODN having chemical modifications from native nucleic acids, or nucleic acid constructs that express such anti-sense molecules as RNA. The antisense sequence is complementary to one or more regions of the targeted Site C protein mRNA, and is capable of reducing or inhibiting the expression of the targeted Site C protein. In general, antisense molecules inhibit expression through various mechanisms, e.g. by reducing the amount of mRNA available for translation, through activation of RNAse H, or steric hindrance. One or a combination of antisense molecules may be administered, where a combination may comprise multiple different sequences.

Antisense molecules may be produced by expression of all or a part of the target gene sequence in an appropriate vector, where the transcriptional initiation is oriented such that an antisense strand is produced as an RNA molecule. Alternatively, the antisense molecule is a synthetic oligonucleotide. Antisense oligonucleotides will generally be at least about 7, usually at least about 12, more usually at least about 20 nucleotides in length, and not more than about 500, usually not more than about 50, more usually not more than about 35 nucleotides in length, where the length is governed by efficiency of inhibition, specificity, including absence of cross-reactivity, and the like. It has been found that short oligonucleotides, of from 7 to 8 bases in length, can be strong and selective inhibitors of gene expression (see Wagner et al. (1996), Nature Biotechnol. 14:840-844).

A specific region or regions of the endogenous sense strand mRNA sequence is chosen to be complemented by the antisense sequence. Selection of a specific sequence for the oligonucleotide may use an empirical method, where several candidate sequences are assayed for inhibition of expression of the target gene in an in vitro or animal model. A combination of sequences may also be used, where several regions of the mRNA sequence are selected for antisense complementation.

Antisense oligonucleotides may be chemically synthesized by methods known in the art (see Wagner et al. (1993), supra.) Preferred oligonucleotides are chemically modified from the native phosphodiester structure, in order to increase their intracellular stability and binding affinity. A number of such modifications have been described in the literature, which alter the chemistry of the backbone, sugars or heterocyclic bases.

Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH₂-5′-O-phosphonate and 3′-NH-5′-O-phosphoroamidate. Peptide nucleic acids replace the entire ribose phosphodiester backbone with a peptide linkage. Sugar modifications are also used to enhance stability and affinity. The α-anomer of deoxyribose may be used, where the base is inverted with respect to the natural β-anomer. The 2′-OH of the ribose sugar may be altered to form 2′-O-methyl or 2′-O-allyl sugars, which provides resistance to degradation without comprising affinity. Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

As an alternative to anti-sense inhibitors, catalytic nucleic acid compounds, e.g. ribozymes, anti-sense conjugates, etc. may be used to reduce or inhibit expression. Ribozymes may be synthesized in vitro and administered to the patient, or may be encoded on an expression vector, from which the ribozyme is synthesized in the targeted cell (for example, see International patent application WO 9523225, and Beigelman et al. (1995), Nucl. Acids Res. 23:4434-42). Examples of oligonucleotides with catalytic activity are described in WO 9506764. Conjugates of anti-sense ODN with a metal complex, e.g. terpyridylCu(II), capable of mediating mRNA hydrolysis are described in Bashkin et al. (1995), Appl. Biochem. Biotechnol. 54:43-56.

In still another embodiment, the Site C regulator of the present invention includes any agent or entity capable of regulating the activation or inactivation of a Site C protein. For example, the Site C regulator of the present invention can be any agent, e.g., a kinase or phosphotase capable of regulating the phosphorylation or dephosphorylation of a Site C protein. In one embodiment, serine 291 of LSF as described in Pagon et al., (Pagon et al., Journal of Cellular Biochemistry 89:733-746 (2003)) can be used as a site for regulating the phosphorylation state of LSF.

A feature of certain embodiments of subject invention is that modulation is achieved by modulating the interaction of a Site C domaine and a Site C repressor protein complex. In these embodiments, the Site C repressor protein complex whose activity is targeted in the subject methods is a protein complex that is made up of one or more proteins, where the protein complex may include a single protein or a plurality of two or more proteins, e.g., 2, 3, 4, 5 or more proteins. A feature of the target repressor protein complex is that it includes an LSF protein.

In these embodiments, the target Site C repressor protein complex whose interaction with the Site C repressor site is modulated in the subject methods is a protein complex made up of one or more proteins that binds to the Site C repressor site and, in so binding, inhibits TERT expression. In certain of these embodiments, the target Site C repressor protein complex includes an LSF protein, as described above.

In certain embodiments, the target repressor protein complex is made up of a single protein, where this protein is an LSF protein, where in certain embodiments the protein is a human LSF protein, or a protein that is substantially similar or identical thereto, as determined using sequence comparison tools described elsewhere in this specification.

In certain embodiments, the target repressor protein complex includes two or more proteins, one of which is an LSF protein as described above. In these embodiments, other protein members of the complex may include, but are not limited to, the repressor proteins described in application Ser. Nos. 10/177,744 and PCT/US02/07918 and 60/323,358; the disclosures of which are herein incorporated by reference.

As mentioned above, in certain embodiments, the target repressor protein complex includes a protein that is substantially the same as one of the above specifically provided proteins, e.g., an LSF protein. By “substantially the same as” is meant a protein having a sequence that has at least about 50%, usually at least about 60% and more usually at least about 75%, and in many embodiments at least about 80%, usually at least about 90% and more usually at least about 95%, 96%, 97%, 98% or 99% sequence identity with the sequence of the above provided sequences, as measured by the BLAST compare two sequences program available on the NCBI website using default settings.

In addition to the specific repressor proteins described above, homologs or proteins (or fragments thereof) from other species, i.e., other animal species, are also of interest, where such homologs or proteins may be from a variety of different types of species, usually mammals, e.g., rodents, such as mice, rats; domestic animals, e.g. horse, cow, dog, cat; and primates, e.g., monkeys, baboons, humans etc. By homolog is meant a protein having at least about 35%, usually at least about 40% and more usually at least about 60% amino acid sequence identity to the specific human transcription repressor factors as identified above, where sequence identity is determined using the algorithm described supra.

In certain embodiments, the target Site C repressor protein complex acts in concert with one or more additional cofactors in binding to the Site C repressor site to inhibit the TERT transcription site. For example, in certain embodiments the Site C repressor protein complex's repressive activity upon binding to the Site C sequence is modulated by its interaction with one or more additional cofactors.

As indicated above, in modulating TERT expression, the interaction between the Site C repressor site and its target repressor protein complex can be modified directly or indirectly. An example of direct modification of this interaction is where the binding of the repressor protein complex to the target sequence is modified by an agent that directly changes how the repressor protein complex binds to the Site C sequence, e.g., by occupying the DNA binding site of the repressor protein complex, by binding to the Site C sequence thereby preventing its binding to the repressor protein complex, etc. An example of indirect modification is modification/modulation of the Site C repressive activity via disruption of a binding interaction between the repressor protein complex and one or more cofactors (or further upstream in the chain of interactions, such as cofactors that interact with the Site C binding protein to make it either a repressor or activator, as described above) such that the repressive activity is modulated, by modification/alteration of the Site C DNA binding sequence such that binding to the repressor protein is modulated, etc.

In certain embodiments, the methods are methods of enhancing TERT expression. By enhancing TERT expression is meant that the expression level of the TERT coding sequence is increased by at least about 2-fold, usually by at least about 5-fold and sometimes by at least about 25-, about 50-, about 100-fold and in particular about 300-fold or higher, as compared to a control, i.e., expression from an expression system that is not subjected to the methods of the present invention. Alternatively, in cases where expression of the TERT gene is so low that it is undetectable, expression of the TERT gene is considered to be enhanced if expression is increased to a level that is easily detectable.

In these methods, Site C repression of TERT expression is inhibited. By inhibited is meant that the repressive activity of the TERT Site C repressor binding site/repressor protein complex interaction with respect to TERT expression is decreased by a factor sufficient to at least provide for the desired enhanced level of TERT expression, as described above. Inhibition of Site C transcription repression may be accomplished in a number of ways, where representative protocols for inhibiting this TERT expression repression are now provided.

One representative method of inhibiting repression of transcription is to employ double-stranded, i.e., duplex, oligonucleotide decoys for the Site C repressor protein complex, as described above. In certain embodiments, the length of these duplex oligonucleotide decoys ranges from about 5 to about 5000, such as from about 5 to about 500 and including from about 10 to about 50 bases.

Instead of the above-described decoys, other agents that disrupt binding of the Site C repressor protein complex to the target TERT Site C repressor binding site and thereby inhibit its expression repression activity may be employed. Other agents of interest include, among other types of agents, small molecules that bind to the Site C repressor protein complex and inhibit its binding to the Site C repressor region. Alternatively, agents that bind to the Site C sequence and inhibit its binding to the Site C repressor protein complex are of interest. Alternatively, agents that disrupt Site C repressor protein complex protein-protein interactions with cofactors, e.g., cofactor binding, and thereby inhibit Site C repression are of interest.

Naturally occurring or synthetic small molecule compounds of interest include numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Such molecules may be identified, among other ways, by employing the screening protocols described below. Small molecule agents of particular interest include pyrrole-imidazole polyamides, analogous to those described in Dickinson et al., Biochemistry Aug. 17, 1999;38(33):10801-7. Other agents include “designer” DNA binding proteins that bind Site C (without causing repression) and prevent the Site C repressor protein complex from binding.

In yet other embodiments, expression of at least one member, e.g., an LSF protein, of the Site C repressor protein complex is inhibited. Inhibition of Site C repressor protein expression may be accomplished using any convenient means, including use of an agent that inhibits Site C repressor protein complex member expression (e.g., antisense agents, RNAi agents, agents that interfere with transcription factor binding to a promoter sequence of the target Site C repressor protein gene, etc,), inactivation of the Site C repressor protein complex member gene, e.g., through recombinant techniques, etc.

For example, antisense molecules can be used to down-regulate expression of the target repressor protein in cells, where representative anti-sense molecules are described above. As an alternative to anti-sense inhibitors, catalytic nucleic acid compounds, e.g. ribozymes, anti-sense conjugates, etc. may be used to inhibit gene expression, as described above.

In another embodiment, the Site C repressor protein complex member gene is inactivated so that it no longer expresses a functional repressor protein. By inactivated is meant that the Site C repressor protein complex member gene, e.g., coding sequence and/or regulatory elements thereof, is genetically modified so that it no longer expresses functional repressor protein complex member, e.g., a functional LSF protein, as described above.

The above-described methods of enhancing TERT expression find use in a number of different applications. In many applications, the subject methods and compositions are employed to enhance TERT expression in a cell that endogenously comprises a TERT gene, e.g., for enhancing expression of hTERT in a normal human cell in which TERT expression is repressed. The target cell of these applications is, in many instances, a normal cell, e.g. a somatic cell. Expression of the TERT gene is considered to be enhanced if, consistent with the above description, expression is increased by at least about 2-fold, usually at least about 5-fold and often at least about 25-, about 50-, about 100-fold, about 300-fold or higher, as compared to a control, e.g., an otherwise identical cell not subjected to the subject methods, or becomes detectable from an initially undetectable state, as described above.

A more specific application in which the subject methods find use is to increase the proliferative capacity of a cell. The term “proliferative capacity” as used herein refers to the number-of divisions that a cell can undergo, and preferably to the ability of the target cell to continue to divide where the daughter cells of such divisions are not transformed, i.e., they maintain normal response -to growth and cell cycle regulation. The subject methods typically result in an increase in proliferative capacity of at least about 1.2-2 fold, usually at least about 5 fold and often at least about 10, about 20, about 50 fold or even higher, compared to a control. As such, yet another more specific application in which the subject methods find use is in the delay of the occurrence of cellular senescence. By practicing the subject methods, the onset of cellular senescence may be delayed by a factor of at least about 1.2-2 fold, usually at least about 5 fold and often at least about 10, about 20, about 50 fold or even higher, compared to a control.

As mentioned above, also provided are methods for inhibiting TERT expression, e.g., by enhancing Site C repression of TERT expression and thereby inhibiting TERT expression. In such methods, the amount and/or activity of the target Site C repressor protein complex is increased so as to enhance Site C repressor mediated repression of TERT expression. A variety of different protocols may be employed to achieve this result, including administration of an effective amount of the Site C repressor protein complex or analog/mimetic thereof (or one or more members thereof), an agent that enhances expression of at least one member of the Site C repressor protein-complex or an agent that enhances the activity of the Site C repressor protein complex.

As such, the nucleic acid compositions that encode the one or more members of the Site C repressor protein complex find use in situations where one wishes to enhance the activity of the repressor protein complex members in a host. The repressor protein genes, gene fragments, or the encoded proteins or protein fragments are useful in gene therapy to treat disorders in which inhibition of TERT expression is desired, including those applications described in greater detail below. Expression vectors may be used to introduce the gene into a cell. Such vectors generally have convenient restriction sites located near the promoter sequence to provide for the insertion of nucleic acid sequences. Transcription cassettes may be prepared comprising a transcription initiation region, the target gene or fragment thereof, and a transcriptional termination region. The transcription cassettes may be introduced into a variety of vectors, e.g. plasmid; retrovirus, e.g. lentivirus; adenovirus; and the like, where the vectors are able to transiently or stably be maintained in the cells, usually for a period of at least about one day, more usually for a period of at least about several days to several weeks.

The gene or protein may be introduced into tissues or host cells by any number of routes, including viral infection, microinjection, or fusion of vesicles. Jet injection may also be used for intramuscular administration, as described by Furth et al. (1992), Anal Biochem 205:365-368. The DNA may be coated onto gold microparticles, and delivered intradermally by a particle bombardment-device, or. “gene gun” as described in the literature (see, for example, Tang et al. (1992), Nature 356:152-154), where gold microprojectiles are coated with the DNA, then bombarded into skin cells.

According to another feature of the present invention, it provides methods of regulating the replicative capacity of a cell by regulating the promoter activity of TERT. According to the present invention, the replicative capacity of a cell can be regulated by contacting the cell with a TERT promoter regulator, e.g., Site C regulator, which modulates the activity of TERT promoter whereby modulating the activity of TERT, thus the replicative capacity of the cell. In general, the replicative capacity of a cell means the potential number of replication events a cell can provide, e.g., number of population doublings. Methods and procedures for determining the replicative capacity of a cell are well known to one skilled in the art.

For example, one can determine the replicative capacity of a cell by incubating the cell in a tissue culture and dividing up the cell population every time it reaches confluency. Usually the number of times a cell population can be divided before it reaches senescence after a division, e.g., when it takes at least 10 times longer than usual for the cell culture to reach confluency represents the replicative capacity of the cell. In particular, the following steps can be used to determine the replicative capacity of a cell population: 1) collecting half of the cell population in a confluent culture flask, 2) inoculating another culture flask of the same size as in step 1) with the collected cell population and allowing the cell culture to reach confluency (typically about 30 hours), 3) repeat the steps of 1) and 2), until it takes at least 10 times longer than usual for the cell culture to reach confluency, e.g., when it takes two weeks for the cell culture to reach confluency, and 4) counting the number of times the process has been repeated, wherein such number represents the replicative capacity of the cell population.

According to the present invention, the replicative capacity of various cell types can be regulated by regulating the activity of TERT. In general, these cells include cells associated with none, few, inter-medium, or high number of replication events. Examples of these cell types include, without any limitation, skin cells, e.g., keratinocytes, melanocytes, hair follicle, and fibroblasts, endothelial cells, e.g., vascular endothelial cells, epithelial cells, e.g., bronchial epithelial cells and retinal pigment epithelial cells, cells associated with joints, e.g., chondrocytes, immune cells, e.g., B cells, T cells, and macrophages, hepatocytes, hematopoietic cells, hematopoietic stem cells, neurons, astrocytes, gastrointestinal cells, renal cells, e.g., renal tubular cells, cells associated with bone formation and structure, e.g. osteoblasts, osteocytes, and osteoclasts, germ cells, muscle cells, e.g., skeletal muscle cells, smooth muscle cells, cardiac myocytes, and neoplastic cells, e.g., cancer and tumor cells.

The methods find use in a variety of therapeutic applications in which it is desired to modulate, e.g., increase or decrease, TERT expression in a target cell or collection of cells, where the collection of cells may be a whole animal or portion thereof, e.g., tissue, organ, etc. As such, the target cell(s) may be a host animal or portion thereof, or may be a therapeutic cell (or cells) which is to be introduced into a multicellular organism, e.g., a cell employed in gene therapy.

As such, embodiments of the invention provide methods for treating various conditions associated with the activity of TERT by administering to a subject, e.g., mammal such as human in need of such treatment an agent capable of regulating the promoter activity of TERT, e.g., a Site C regulator. Conditions associated with the activity of TERT include any condition associated with the expression, activation, quantitative and qualitative level of TERT and any condition associated with telomeres, e.g., the length of telomeres. In general, such condition is associated with aging, neoplastic growth, or related to cell replication or turn over, e.g., conditions associated with high cell replication event or turn over. For example, conditions associated with the activity of TERT include progeria, atherosclerosis, cardiovascular diseases, osteoarthritis, osteoporosis, Alzheimer's disease, macular degeneration, liver cirrhosis, rheumatoid arthritis, AIDS or HIV infection, autoimmune disease, muscular dystrophy, wound healing, hair loss, photo-damaged skin, transplantation, cancer, and tumor. Usually most conditions are associated with decreased level or absence of TERT activity whereas in neoplastic growth, the condition is associated with the presence or increased level of TERT activity.

In such methods, an effective amount of an active agent that modulates TERT expression, e.g., enhances or decreases TERT expression as desired, is administered to the target cell or cells, e.g., by contacting the cells with the agent, by administering the agent to the animal, etc. By effective amount is meant a dosage sufficient to modulate TERT expression in the target cell(s), as desired.

In the subject methods, the active agent(s) may be administered to the targeted cells using any convenient means capable of resulting in the desired enhancement of TERT expression. Thus, the agent can be incorporated into a variety of formulations for therapeutic administration. More particularly, the agents of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments (e.g., skin creams), solutions, suppositories, injections, inhalants and aerosols. As such, administration of the agents can be achieved in various ways, including oral, buccal, rectal, parenteral, intraperitoneal, intradermal, transdermal, intracheal, etc., administration.

In pharmaceutical dosage forms, the agents may be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The agents can be utilized in aerosol formulation to be administered via inhalation. The compounds of the present invention can be formulated into pressurized acceptable propellants such as dichlorodifluoromethane, propane, nitrogen and the like.

Furthermore, the agents can be made into suppositories by mixing with a variety of bases such as emulsifying bases or water-soluble bases. The compounds of the present invention-can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Where the agent is a polypeptide, polynucleotide, analog or mimetic thereof, e.g. oligonucleotide decoy, it may be introduced into tissues or host cells by any number of routes, including viral infection, microinjection, or fusion of vesicles. Jet injection may also be used for intramuscular administration, as described by Furth et al. (1992), Anal Biochem 205:365-368. The DNA may be coated onto gold microparticles, and delivered intradermally by a particle bombardment device, or “gene gun” as described in the literature (see, for example, Tang et al. (1992), Nature 356:152-154), where gold microprojectiles are coated with the DNA, then bombarded into skin cells. For nucleic acid therapeutic agents, a number of different delivery vehicles find use, including viral and non-viral vector systems, as are known in the art.

In one embodiment, the active agent is prepared with carriers that will protect the agent against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters and polylactic acid.

Methods for preparation of such formulations will be apparent to those skilled in the art. The materials also can be obtained commercially from Alza Corporation and Nova Pharmaceuticals, Inc.

Liposomal suspensions (including liposomes targeted to infected cells with monoclonal antibodies) also can be used as pharmaceutically acceptable carriers. Those can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The dosages, for example, preferred route of administration and amounts, are obtainable based on empirical data obtained from preclinical and clinical studies, practicing methods known in the art. For repeated administrations over several days or longer, depending on the condition, the treatment is sustained until a desired suppression of disease symptoms occurs. However, other dosage regimens may be useful. The progress of the therapy is monitored easily by conventional techniques and assays. An exemplary dosing regimen is disclosed in WO 94/04188. The specification for the dosage unit forms of the invention is dictated by and directly dependent on the unique characteristics of the active compound and the particular therapeutic effect to be achieved and the limitations inherent in the art of compounding such an active compound for the treatment of individuals.

The pharmaceutical compositions can be included in a container, pack or dispenser together with instructions for administration.

Another method of administration comprises the addition of a composition of interest into or with a food or drink, as a food supplement or additive, or as a dosage form taken on a prophylactic basis, similar to a vitamin. The peptide or nucleotide of interest can be encapsulated into forms that will survive passage through the gastric environment. Such forms are commonly known as enteric-coated formulations. Alternatively, the peptide or nucleotide of interest can be modified to enhance half-life, such as chemical modification of the peptide or nucleotide bonds, to ensure stability for oral administration, as known in the art.

The pharmaceutical compositions of the present invention employed in the above methods include at least one or more agents of the present invention, e.g., a Site C regulator and a carrier. In one embodiment, the pharmaceutical composition of the present invention includes at least one Site C regulator. In another embodiment, the pharmaceutical composition of the present invention includes at least two Site C regulators, e.g., including at least one Site C protein. In yet another embodiment, the pharmaceutical composition of the present invention includes at least two Site C proteins, e.g. capable of forming a complex to interact with one or more repressor sites associated with TERT promoter. For example, the pharmaceutical composition of the present invention can include at least two members of LSF family, e.g., LBP1a and LBP1c or LBP1b and LBP1c. Alternatively, the pharmaceutical composition of the present invention can include at least three members of LSF family, e.g., LBP1a, LBP1c, and LBP9 or LBP1b, LBP1c and LBP9. In general, a Site C protein in a pharmaceutical composition can be a polypeptide or polynucleotide in a vector suitable for expressing the Site C protein.

Those of skill in the art will readily appreciate that dose levels can vary as a function of the specific compound, the nature of the delivery vehicle, and the like. Preferred dosages for a given compound are readily determinable by those of skill in the art by a variety of means.

The subject methods find use in the treatment of a variety of different conditions in which the modulation, e.g., enhancement or decrease, of TERT expression in the host is desired. By treatment is meant that at least an amelioration of the symptoms associated with the condition afflicting the host is achieved, where amelioration is used in a broad sense to refer to at least a reduction in the magnitude of a parameter, e.g. symptom (such as inflammation), associated with the condition being treated. As such, treatment also includes situations where the pathological condition, or at least symptoms associated therewith, are completely inhibited, e.g. prevented from happening, or stopped, e.g. terminated, such that the host no longer suffers from the condition, or at least the symptoms that characterize the condition.

A variety of hosts are treatable according to the subject methods. Generally such hosts are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the hosts will be humans.

As indicated above, the subject invention provides methods of treating disease conditions resulting from a lack of TERT expression and methods of-treating disease conditions resulting from unwanted TERT expression. Representative disease conditions for each category are now described in greater detail separately.

One representative disease condition that may be treated according to the subject invention is Progeria, or Hutchinson-Gilford syndrome. This condition is a disease of shortened telomeres for which no known cure exists. It afflicts children, who seldom live past their early twenties. In many ways progeria parallels aging itself. However, these children are born with short telomeres. Their telomeres do not shorten at a faster rate; they are just short to begin with. The subject methods can be used in such conditions to further delay natural telomeric shortening and/or increase telomeric length, thereby treating this condition.

Another specific disease condition in which the subject methods find use is in immune senescence. The effectiveness of the immune system decreases with age. Part of this decline is due to fewer T-lymphocytes in the system, a result of lost replicative capacity. Many of the remaining T-lymphocytes experience loss of function as their telomeres shorten and they approach senescence. The subject methods can be employed to inhibit immune senescence due to telomere loss. Because hosts with aging immune systems are at greater risk of developing pneumonia, cellulitis, influenza, and many other infections, the subject methods reduce morbidity and mortality due to infections.

The subject methods also find use in AIDS therapy. HIV, the virus that causes AIDS, invades white blood cells, particularly CD4 lymphocyte cells, and causes them to reproduce high numbers of the HIV virus, ultimately killing cells. In response to the loss of immune cells (typically about a billion per day), the body produces more CD8 cells to be able to suppress infection. This rapid cell division accelerates telomere shortening, ultimately hastening immune senescence of the CD8 cells. Anti-retroviral therapies have successfully restored the immune systems of AIDS patients, but survival depends upon the remaining fraction of the patient's aged T-cells. Once shortened, telomere length has not been naturally restored within cells. The subject methods can be employed to restore this length and/or prevent further shortening. As such the subject methods can spare telomeres and is useful in conjunction with the anti-retroviral treatments currently available for HIV.

Yet another type of disease condition in which the subject methods find use is cardiovascular disease. The subject methods can be employed to extend telomere length and replicative capacity of endothelial cells lining blood vessel walls (DeBono, Heart 80:110-1, 1998). Endothelial cells form the inner lining of blood vessels and divide and replace themselves in response to stress. Stresses include high blood pressure, excess cholesterol, inflammation, and flow stresses at forks in vessels. As endothelial cells age and can no longer divide sufficiently to replace lost cells, areas under the endothelial layer become exposed. Exposure of the underlying vessel wall increases inflammation, the growth of smooth muscle cells, and the deposition of cholesterol. As a result, the vessel narrows and becomes scarred and irregular, which contributes to even more stress on the vessel (Cooper, Cooke and Dzau, J Gerontol Biol Sci 49: 191-6, 1994). Aging endothelial cells also produce altered amounts of trophic factors (hormones that affect the activity of neighboring cells). These too contribute to increased clotting, proliferation of smooth muscle cells, invasion by white blood cells, accumulation of cholesterol, and other changes, many of which lead to plaque formation and clinical cardiovascular disease (Ibid.). By extending endothelial cell telomeres, the subject methods can be employed to combat the stresses contributing to vessel disease. Many heart attacks may be prevented if endothelial cells were enabled to continue to divide normally and better maintain cardiac vessels. The occurrence of strokes caused by the aging of brain blood vessels may also be significantly reduced by employing the subject methods to help endothelial cells in the brain blood vessels to continue to divide and perform their intended function.

The subject methods also find use in skin rejuvenation. The skin is the first line of defense of the immune system and shows the most visible signs of aging (West, Arch Dermatol 130(1):87-95, 1994). As skin ages, it thins, develops wrinkles, discolors, and heals poorly. Skin cells divide quickly in response to stress and trauma; but, over time, there are fewer and fewer actively dividing skin cells. Compounding the loss of replicative capacity in aging skin is a corresponding loss of support tissues. The number of blood vessels in the skin decreases with age, reducing the nutrients that reach the skin. Also, aged immune cells less effectively fight infection. Nerve cells have fewer branches, slowing the response to pain and increasing the chance of trauma. In aged skin, there are also fewer fat cells, increasing susceptibility to cold and temperature changes. Old skin cells respond more slowly and less accurately to external signals. They produce less vitamin D, collagen, and elastin, allowing the extracellular matrix to deteriorate. As skin thins and loses pigment with age, more ultraviolet light penetrates and damages skin. To repair the increasing ultraviolet damage, skin cells need to divide to replace damaged cells, but aged skin cells have shorter telomeres and are less capable of dividing (Fossel, REVERSING HUMAN AGING. William Morrow & Company, New York City, 1996).

By practicing the subject methods, e.g., via administration of an active agent topically, one can extend telomere length, and slow the downward spiral that skin experiences with age. Such a product not only helps protect a person against the impairments of aging skin; it also permits rejuvenated skin cells to restore youthful immune resistance and appearance. The subject methods can be used for both medical and cosmetic skin rejuvenation applications.

Yet another disease condition in which the subject methods find use in the treatment of osteoporosis. Two types of cells interplay in osteoporosis: osteoblasts make bone and osteoclasts destroy it. Normally, the two are in balance and maintain a constant turnover of highly structured bone. In youth, bones are resilient, harder to break, and heal quickly. In old age, bones are brittle, break easily, and heal slowly and often improperly. Bone loss has been postulated to occur because aged osteoblasts, having lost much of their replicative capacity, cannot continue to divide at the rate necessary to maintain balance (Hazzard et al. PRINCIPLES OF GERIATRIC MEDICINE AND GERONTOLOGY, 2d ed. McGraw-Hill, New York City, 1994). The subject methods can be employed to lengthen telomeres of osteoblast and osteoclast stem cells, thereby encouraging bone replacement and proper remodeling and reinforcement. The resultant stronger bone improves the quality of life for the many sufferers of osteoporosis and provides savings from fewer fracture treatments. The subject methods are generally part of a comprehensive treatment regime that also includes calcium, estrogen, and exercise.

Additional disease conditions in which the subject methods find use are described in WO 99/35243, the disclosures of which are herein incorporated by reference.

In addition to the above-described methods, the subject methods can also be used to extend the lifetime of a mammal. By extend the lifetime is meant to increase the time during which the animal is alive, where the increase is generally at least 1%, usually at least 5% and more usually at least about 10%, as compared to a control. As indicated above, instead of a multicellular animal, the target may be a cell or population of cells which are treated according to the subject methods and then introduced into a multicellular organism for therapeutic effect. For example, the subject methods may be employed in bone marrow transplants for the treatment of cancer and skin grafts for burn victims. In these cases, cells are isolated from a human donor and then cultured for transplantation back into human recipients. During the cell culturing, the cells normally age and senesce, decreasing their useful lifespans. Bone marrow cells, for instance, lose approximately 40% of their replicative capacity during culturing. This problem is aggravated when the cells are first genetically engineered (Decary, Mouly et al. Hum Gene Ther 7(11): 1347-50, 1996). In such cases, the therapeutic cells must be expanded from a single engineered cell. By the time there are sufficient cells for transplantation., the cells have undergone the equivalent of 50 years of aging (Decary, Mouly et al. Hum Gene Ther 8(12): 1429-38, 1997). Use of the subject methods spares the replicative capacity of bone marrow cells and skin cells during culturing and expansion and thus significantly improves the survival and effectiveness of bone marrow and skin cell transplants. Any transplantation technology requiring cell culturing can benefit from the subject methods, including ex vivo gene therapy applications in which cells are cultured outside of the animal and then administered to the animal, as described in U.S. Pat. Nos. 6,068,837; 6,027,488; 5,824,655; 5,821,235; 5,770,580; 5,756,283; 5,665,350; the disclosures of which are herein incorporated by reference.

As summarized above, also provided are methods for enhancing repression of TERT expression, where by enhancement of TERT expression repression is meant a decrease in TERT expression by a factor of at least about 2-fold, usually at least about 5-fold and more usually at least about 10-fold, as compared to a control. Methods for enhancing Site C mediated repression of TERT expression find use in, among other applications, the treatment of cellular proliferative disease conditions, particularly abnormal cellular proliferative disease conditions, including, but not limited to, neoplastic disease conditions, e.g., cancer. In such applications, an effective amount of an active agent, e.g., a Site C repressor protein complex, analog or mimetic thereof, a vector encoding a Site C repressor protein complex member or members or active fragments thereof, an agent that enhances endogenous Site C repressor protein complex activity, an agent that enhances expression of one or more members of the Site C repressor protein complex, etc., is administered to the subject in need thereof. Treatment is used broadly as defined above, e.g., to include at least an amelioration in one or more of the symptoms of the disease, as well as a complete cessation thereof, as well as a reversal and/or complete removal of the disease condition, e.g., cure. Methods of treating disease conditions resulting from unwanted TERT expression, such as cancer and other diseases characterized by the presence of unwanted cellular proliferation, are described in, for example, U.S. Pat. Nos. 5,645,986; 5,656,638; 5,703,116; 5,760,062; 5,767,278; 5,770,613; and 5,863,936; the disclosures of which are herein incorporated by reference.

### Telemerase Repressor Polypeptide Compositions

The subject invention further provides, in certain embodiments, *telomerase repressor polypeptides, i.e., polypeptides that repress telomerase expression, and specifically LBP1c2 (SEQ ID NO:01 or SEQ ID NO:03) and BOMv2 (SEQ ID NO:04. The term “polypeptide composition” as used herein refers to both full-length proteins as well as portions or fragments thereof. Also included in this term are variations of the naturally occurring proteins, where such variations are homologous or substantially similar to the naturally occurring protein, as described in greater detail below, be the naturally occurring protein the human protein or a protein from some other species that naturally expresses repressor protein, usually a mammalian species. In the following description of the subject invention, the name for a given repressor protein is used to refer not only to the human form of the protein, but also to homologs thereof expressed in non-human species, e.g., murine, rat, monkey and other mammalian species.

The subject repressor proteins are characterized by having TERT repressor activity. Specifically, the subject proteins bind to a repressor binding site present in the TERT minimal promoter. More specifically, the subject proteins bind to a “Site C” repressor binding site present in the human TERT minimal promoter, as described above. When binding to this site, or a portion thereof, the subject repressor proteins inhibit expression of TERT, where by inhibit expression is meant that expression of TERT is reduced by at least about 50%, usually at least about 75% and more usually at least about 90% as compared to a control system where TERT expression occurs and that is identical but for the absence of the subject repressor protein. The subject repressor proteins may be glycosylated, or modified in alternative-ways.

Of particular interest in certain embodiments are the above specified LBP1c2 and BOMv2 proteins, or proteins that are substantially the same as these proteins.

By “substantially the same as” is meant a protein having a sequence that has at least about 50%, usually at least about 60% and more usually at least about 75%, and in many embodiments at least about 80%, usually at least about 90% and more usually at least about 95%, 96%, 97%, 98% or 99% sequence identity with the sequence of the above provided sequences, as measured by the BLAST compare two sequences program available on the NCBI website using default settings.

In addition to the specific TERT repressor proteins described above, homologs or proteins (or fragments thereof from other species, i.e., other animal species, are also provided, where such homologs or proteins may be from a variety of different types of species, usually mammals, e.g., rodents, such as mice, rats; domestic animals, e.g. horse, cow, dog, cat; and primates, e.g., monkeys, baboons, humans etc. By homolog is meant-a protein having at least about 35%, usually at least about 40% and more usually at least about 60% amino acid sequence identity to the specific human transcription repressor factors as identified above, where sequence identity is determined using the algorithm described supra.

The TERT repressor proteins of the subject invention are present in a non-naturally occurring environment, e.g., are separated from their naturally occurring environment. In certain embodiments, the subject proteins are present in a composition that is enriched for the subject proteins as compared to the subject proteins in their naturally occurring environment. As such, purified repressor proteins according to the subject invention are provided, where by purified is meant that the proteins are present in a composition that is substantially free of non repressor proteins of the subject invention, where by substantially free is meant that less than 90%, usually less than 60% and more usually less than 50% of the composition is made up of non-repressor proteins of the subject invention.

In certain embodiments of interest, the repressor proteins are present in a composition that is substantially free of the constituents that are present in its naturally occurring environment. For example, a human repressor protein comprising composition according to the subject invention in this embodiment will be substantially, if not completely, free of those other biological constituents, such as proteins, carbohydrates, lipids, etc., with which it is present in its natural environment. As such, protein compositions of these embodiments will necessarily differ from those that are prepared by purifying the protein from a naturally occurring source, where at least trace amounts of the constituents or other components of the protein's naturally occurring source will still be present in the composition prepared from the naturally occurring source.

The repressor proteins of the subject invention may also be present as isolates, by which is meant that the proteins are substantially free of both non-repressor proteins and other naturally occurring biologic molecules, such as oligosaccharides, polynucleotides and fragments thereof, and the like, where substantially free in this instance means that less than 70%, usually less than 60% and more usually less than 50% (by dry weight) of the composition containing the isolated repressor proteins is a non-repressor protein naturally occurring biological molecule. In certain embodiments, the repressor proteins are present in substantially pure form, where by substantially pure form is meant at least 95%, usually at least 97% and more usually at least 99% pure.

In addition to the naturally occurring proteins, polypeptides that vary from the naturally occurring proteins are also provided. By polypeptide is meant proteins having an amino acid sequence encoded by an open reading frame (ORF) of a repressor protein gene, described below, including-the full length protein and fragments thereof, particularly biologically active fragments and/or fragments corresponding to functional domains, and including fusions of the subject polypeptides to other proteins or parts thereof, e.g., immunoglobulin domains, nuclear localization domains (such as a VP22 domain as described in U.S. Pat. No. 6,358,739, the disclosure of which is herein incorporated by reference); and the like. Fragments of interest will typically be at least about 10 aa in length, usually at least about 50 aa in length, and may be as long as 300 aa in length or longer, but will usually not exceed about 1000 aa in length.

Also provided by the subject invention are ligands having TERT Site C binding activity. The term ligand, as used herein, refers to any compound capable of binding to a TERT repressor site, particularly Site C, and as such includes proteins and peptides, oligosaccharides, and the like, as well as binding mimetics thereof, including small molecule binding mimetics thereof. The subject ligands are capable of binding to Site C in a manner analogous to the binding activity of the subject repressor proteins, and will generally comprise the functional TERT promoter binding domain, e.g., Site C binding domain, of a repressor protein according to the subject invention, or the functional equivalent thereof.

### Nucleic Acid Compositions

Also provided are nucleic acid compositions that encode TERT expression repressor polypeptides and fragments thereof, etc., as described above. Specifically, nucleic acid compositions encoding the subject polypeptides, as well as fragments or homologs thereof, are provided. By “nucleic acid composition” is meant a composition comprising a sequence of nucleotide bases that encodes a polypeptide according to the subject invention, i.e., a region of genomic DNA capable of being transcribed into mRNA that encodes a repressor polypeptide, the mRNA that encodes and directs the synthesis of a repressor polypeptide, etc. Specific nucleic acids of interest include those identified herein as SEQ ID NO:02; and SEQ ID NO:05. Also encompassed in this term are nucleic acids that are homologous, substantially similar or identical to the nucleic acids specifically disclosed herein.

Also provided are nucleic acids that are homologous to the provided nucleic acids, at least with respect to the coding regions thereof. The source of homologous nucleic acids to those specifically listed above may be any mammalian species, e.g., primate species, particularly human; rodents, such as rats and mice, canines, felines, bovines, equines, etc; as well as non-mammalian species, e.g., yeast, nematodes, etc. Between mammalian species, e.g., human and mouse, homologs have substantial sequence similarity, e.g., at least 75% sequence identity, usually at least 90%, more usually at least 95% between nucleotide sequences. Sequence similarity is calculated based on a reference sequence, which may be a subset of a larger sequence, such as a conserved motif, coding region, flanking region, etc. A reference sequence will usually be at least about 18 nt long, more usually at least about 30 nt long, and may extend to the complete sequence that is being compared. Algorithms for sequence analysis are known in the art, such as BLAST, described in Altschul et al. (1990), J. Mol. Biol. 215:403-10 (using default settings, i.e. parameters w=4 and T=17). Unless indicated otherwise, the sequence similarity values reported herein are those determined using the above referenced BLAST program using default settings. The sequences provided herein are essential for recognizing TERT repressor related and homologous polynucleotides in database searches. Of particular interest in certain embodiments are nucleic acids including a sequence substantially similar to the specific nucleic acids identified above, where by substantially similar is meant having sequence identity to this sequence of at least about 90%, usually at least about 95% and more usually at least about 99%.

Also provided are nucleic acids that hybridize to the above described nucleic acids under stringent conditions. An example of stringent hybridization conditions is hybridization at 50° C. or higher and 0.1×SSC (15 mM sodium chloride/1.5 mM sodium citrate). Another example of stringent hybridization conditions is overnight incubation at 42° C. in a solution: 50% formamide, 5×SSC (150 mM NaCl, 15 mM trisodium citrate), 50 mM sodium phosphate (pH7.6), 5× Denhardt's solution, 10% dextran sulfate, and 20 μg/ml denatured, sheared salmon sperm DNA, followed by washing the filters in 0.1×SSC at about 65° C. Stringent hybridization conditions are hybridization conditions that are at least as stringent as the above representative conditions. Other stringent hybridization conditions are known in the art and may also be employed to identify nucleic acids of this particular embodiment of the invention.

Nucleic acids encoding the proteins and polypeptides of the subject invention may be cDNAs or genomic DNAs, as well as fragments thereof. The nucleic acids may also be mRNAs, e.g., transcribed from genomic DNA, that encode (i.e. are translated into) the subject proteins and polypeptides. Also provided are genes encoding the subject proteins, where the term “gene” means the open reading frame encoding specific proteins and polypeptides, and introns that are present in the open reading frame, as well as adjacent 5′ and 3′ non-coding nucleotide sequences involved, e.g., untranslated regions, promoter or other regulatory elements, etc., in the regulation of expression, up to about 20 kb beyond the coding region, but possibly further in either direction. The gene may be introduced into an appropriate vector for extrachromosomal maintenance or for integration into a host genome.

The term “cDNA” as used herein is intended to include all nucleic acids that share the arrangement of sequence elements found in native mature mRNA species, where sequence elements at least include exons. Normally mRNA species have contiguous exons, with the intervening introns, when present, being removed by nuclear RNA splicing, to create a continuous open reading frame encoding a repressor protein according to the subject invention.

A genomic sequence of interest comprises the nucleic acid present between the initiation codon and the stop codon, as defined in the listed sequences, including all of the introns that are normally present in a native chromosome. It may further include specific transcriptional and translational regulatory sequences, such as promoters, enhancers, etc., including about 1 kb, but possibly more, of flanking genomic DNA at either the 5′ and 3′ end of the transcribed region. The genomic DNA may be isolated as a fragment of 100 kbp or smaller; and substantially free of flanking chromosomal sequence. The genomic DNA flanking the coding region, either 3′ or 5′, or internal regulatory sequences as sometimes found in introns, contains sequences required for proper tissue and stage specific expression.

The nucleic acid compositions of the subject invention may encode all or a part of the subject proteins and polypeptides, described in greater detail above. Double or single stranded fragments may be obtained from the DNA sequence by chemically synthesizing oligonucleotides in accordance with conventional methods, by restriction enzyme digestion, by PCR amplification, etc. For the most part, DNA fragments will be of at least 15nt, usually at least 18nt or 25 nt, and may be at least about 50 nt.

The TERT repressor genes of the subject invention are isolated and obtained in substantial purity, generally as other than an intact chromosome. Usually, the DNA will be obtained substantially free of other nucleic acid sequences that do not include a TERT repressor protein sequence or fragment thereof, generally being at least about 50%, usually at least about 90% pure and are typically “recombinant,” i.e. flanked by one or more nucleotides with which it is not normally associated on a naturally occurring chromosome.

In addition to the plurality-of uses described in greater detail in following sections, the subject nucleic acid compositions find use in the preparation of all or a portion of the subject polypeptides, as described above.

Also provided are nucleic acid probes, as well as constructs, e.g., vectors, expression systems, etc., as described more fully below, that include a nucleic acid sequence as described above. Probes of the subject invention are generally fragments of the provided nucleic acid. The probes may be a large or small fragment, generally ranging in length from about 10 to 100 nt, usually from about 15 to 50 nt. In using the subject probes, nucleic acids having sequence similarity are detected by hybridization under low stringency conditions, for example, at 50° C. and 6×SSC (0.9 M sodium chloride/0.09 M sodium citrate)(or analogous conditions) and remain bound when subjected to washing at higher stringency conditions, e.g., 55° C. in 1×SSC (0.15 M sodium chloride/0.015 M sodium citrate) (or analogous conditions). Sequence identity may be determined by hybridization under'stringent conditions, for example, at 50° C. or higher and 0.1×SSC (15 mM sodium chloride/01.5 mM sodium citrate)(or analogous conditions). Nucleic acids having a region of substantial identity to the provided nucleic acid sequences bind to the provided sequences under stringent hybridization conditions. By using probes, particularly labeled probes of DNA sequences, one can isolate homologous or related sequences.

The subject nucleic acids are isolated and obtained in substantial purity, generally as other than an intact chromosome. As such, they are present in other than their naturally occurring environment. Usually, the DNA will be obtained substantially free of other nucleic acid sequences that do not include a repressor sequence or fragment thereof, generally being at least about 50%, usually at least about 90% pure and are typically “recombinant”, i.e. flanked by one or more nucleotides with which it is not normally associated on a naturally occurring chromosome.

The subject nucleic acids may be produced using any convenient protocol, including synthetic protocols, e.g., such as those where the nucleic acid is synthesized by a sequential monomeric approach (e.g., via phosphoramidite chemistry); where subparts of the nucleic acid are so synthesized and then assembled or concatamerized into the final nucleic acid, and the like. Where the nucleic acid of interest has a sequence that occurs in nature, the nucleic acid may be retrieved, isolated, amplified etc., from a natural source using conventional molecular biology protocols.

Also provided are constructs comprising the subject nucleic acid compositions, e.g., those that include a repressor protein coding sequence, inserted into a vector, where such constructs may be used for a number of different applications, including propagation, screening, genome alteration, and the like, as described in greater detail below. Constructs made up-of viral and non-viral vector sequences may be prepared and used, including plasmids, as desired. The choice of vector will depend on the particular application in which the nucleic acid is to be employed. Certain vectors are useful for amplifying and making large amounts of the desired DNA sequence. Other vectors are suitable for expression in cells in culture, e.g., for use in screening assays. Still other vectors are suitable for transfer and expression in cells in a whole animal or person. The choice of appropriate vector is well within the ability of those of ordinary skill in the art. Many such vectors are available commercially. To prepare the constructs, the partial or full-length nucleic acid is inserted into a vector typically by means of DNA ligase attachment to a cleaved restriction enzyme site in the vector. Alternatively, the desired nucleotide sequence can be inserted by homologous recombination in vivo. Typically, homologous recombination is accomplished by attaching regions of homology to the vector on the flanks of the desired nucleotide sequence. Regions of homology are added by ligation of oligonucleotides, or by polymerase chain reaction using primers that include both the region of homology and a portion of the desired nucleotide sequence, for example.

Also provided are expression cassettes that include a coding sequence. By expression cassette is meant a nucleic acid that includes a sequence encoding a subject peptide or protein operably linked to a promoter sequence, where by operably linked is meant that expression of the coding sequence is under the control of the promoter sequence.

### Preparation of Polypeptides According to the Subject Invention

The subject proteins may be obtained using any convenient protocol. As such, they may be obtained from naturally occurring sources or recombinantly produced. Naturally occurring sources of the subject proteins include tissues and portions/fractions, including cells and fractions thereof, e.g., extracts, homogenates etc., that include cells in which the desired protein is expressed.

The subject proteins may also be obtained from synthetic protocols, e.g., by expressing a recombinant gene encoding the subject protein, such as the polynucleotide compositions described above, in a suitable host under conditions sufficient for post-translational modification to occur in a manner that provides the expressed protein with TERT repression activity, e.g., Site C binding activity. For expression, an expression cassette may be employed. The expression cassette or vector will provide a transcriptional and translational initiation region, which may be inducible or constitutive, where the coding region is operably linked under the transcriptional control of the transcriptional initiation region, and under the translational control of the translational initiation region, and a transcriptional and translational termination region. These control regions may be native to a gene of the subject invention, or may be derived from exogenous sources.

Expression cassettes may be prepared comprising a transcription initiation region, the nucleic acid coding sequence or fragment thereof, and a transcriptional termination region. Of particular interest is the use of sequences that allow for the expression of functional epitopes or domains, usually at least about 8 amino acids in length, more usually at least about 15 amino acids in length, to about 25 amino acids, and up to the complete open reading frame of the coding sequence. After introduction of the DNA, the cells containing the construct may be selected by means of a selectable marker, the cells expanded and then used for expression.

The subject proteins and polypeptides may be expressed in prokaryotes or eukaryotes in accordance with conventional ways, depending upon the purpose for expression. For large scale production of the protein, a unicellular organism, such as E. coli, B. subtilis, S. cerevisiae, insect cells in combination with baculovirus vectors, or cells of a higher organism such as vertebrates, particularly mammals, e.g. COS 7 cells, may be used as the expression host cells. In some situations, it is desirable to express the gene in eukaryotic cells, where the encoded protein will benefit from native folding and post-translational modifications. Small peptides can also be synthesized in the laboratory. Polypeptides that are subsets of the complete sequence may be used to identify and investigate parts of the protein important for function.

Specific expression systems of interest include bacterial, yeast, insect cell and mammalian cell derived expression systems. Representative systems from each of these categories is are provided below:

Bacteria. Expression systems in bacteria include those described in Chang et al., Nature (1978) 275:615; Goeddel et al., Nature (1979) 281:544; Goeddel et al., Nucleic Acids Res. (1980) 8:4057; EP 0 036,776; U.S. Pat. No. 4,551,433; DeBoer et al., Proc. Natl. Acad. Sci. (USA) (1983) 80:21-25; and Siebenlist et al., Cell (1980) 20:269.

Yeast. Expression systems in yeast include those described in Hinnen et al., Proc. Natl. Acad. Sci. (USA) (1978) 75:1929; Ito et al., J. Bacteriol. (1983) 153:163; Kurtz et al., Mol. Cell. Biol. (1986) 6:142; Kunze et al., J. Basic Microbiol. (1985) 25:141; Gleeson et al., J. Gen. Microbiol. (1986) 132:3459; Roggenkamp et al., Mol. Gen. Genet. (1986) 202:302; Das et al., J. Bacteriol. (1984) 158:1165; De Louvencourt et al., J. Bacteriol. (1983) 154:737; Van den Berg et al., Bio/Technology (1990) 8:135; Kunze et al., J. Basic Microbiol. (1985) 25:141; Cregg et al., Mol. Cell. Biol. (1985) 5:3376; U.S. Pat. Nos. 4,837,148 and 4,929,555; Beach and Nurse, Nature (1981) 300:706; Davidow et al., Curr. Genet. (1985) 10:380; Gaillardin et al., Curr. Genet. (1985) 10:49; Ballance et al., Biochem. Biophys. Res. Commun. (1983) 112:284-289; Tilburn et al., Gene (1983) 26:205-221; Yelton et al., Proc. Natl. Acad. Sci. (USA) (1984) 81:1470-1474; Kelly and Hynes, EMBO J. (1985) 4:475479; EP 0 244,234; and WO 91/00357.

Insect Cells. Expression of heterologous genes in insects is accomplished as described in U.S. Pat. No. 4,745,051; Friesen et al., “The Regulation of Baculovirus Gene Expression”, in: The Molecular Biology Of Baculoviruses (1986) (W. Doerfler, ed.); EP 0 127,839; EP 0 155,476; and Vlak et al., J. Gen. Virol. (1988) 69:765-776; Miller et al., Ann. Rev. Microbiol. (1988) 42:177; Carbonell et al., Gene (1988) 73:409; Maeda et al., Nature (1985) 315:592-594; Lebacq-Verheyden et al., Mol. Cell. Biol. (1988) 8:3129; Smith et al., Proc. Natl. Acad. Sci. (USA) (1985) 82:8844; Miyajima et al., Gene (1987) 58:273; and Martin et al., DNA (1988) 7:99. Numerous baculoviral strains and variants and corresponding permissive insect host cells from hosts are described in Luckow et al., Bio/Technology (1988) 6:47-55, Miller et al., Generic Engineering (1986) 8:277-279, and Maeda et al., Nature (1985) 315:592-594.

Mammalian Cells. Mammalian expression is accomplished as described in Dijkema et al., EMBO J. (1985) 4:761, Gorman et al., Proc. Natl. Acad. Sci. (USA) (1982) 79:6777, Boshart et al., Cell (1985) 41:521 and U.S. Pat. No. 4,399,216. Other features of mammalian expression are facilitated as described in Ham and Wallace, Meth. Enz. (1979) 58:44, Barnes and Sato, Anal. Biochem. (1980) 102:255, U.S. Pat. Nos. 4,767,704, 4,657,866, 4,927,762, 4,560,655, WO 90/103430, WO 87/00195, and U.S. Pat. No. 30,985.

When any of the above host cells, or other appropriate host cells or organisms, are used to replicate and/or express the polynucleotides or nucleic acids of the invention, the resulting replicated nucleic acid, RNA, expressed protein or polypeptide, is within the scope of the invention as a product of the host cell or organism.

Once the source of the protein is identified and/or prepared, e.g. a transfected host expressing the protein is prepared, the protein is then purified to produce the desired repressor protein comprising composition. Any convenient protein purification procedures may be employed, where suitable protein purification methodologies are described in Guide to Protein Purification, (Deuthser ed.) (Academic Press, 1990). For example, a lysate may be prepared from the original source, e.g. naturally occurring cells or tissues that express the subject repressor proteins or the expression host expressing the subject repressor proteins, and purified using HPLC, exclusion chromatography, gel electrophoresis, affinity chromatography, and the like.

Once the gene corresponding to a selected polynucleotide is identified, its expression can be regulated in the cell to which the gene is native. For example, an endogenous gene of a cell can be regulated by an exogenous regulatory sequence as disclosed in U.S. Pat. No. 5,641,670; the disclosure of which is herein incorporated by reference.

### Antibodies

Also provided are antibodies that bind to the subject proteins and homologs thereof. Suitable antibodies are obtained by immunizing a host animal with peptides comprising all or a portion of the repressor protein. Suitable host animals include rat, sheep, goat, hamster, rabbit, etc. The origin of the protein immunogen may be mouse, rat, monkey etc. The host animal will generally be a different species than the immunogen, e.g. human protein used to immunize rabbit, etc.

The immunogen may comprise the complete protein, or fragments and derivatives thereof. Preferred immunogens comprise all or a part of the subject repressor protein, where these residues contain the post-translation modifications, such as glycosylation, found on the native target protein. Immunogens comprising the extracellular domain are produced in a variety of ways known in the art, e.g. expression of cloned genes using conventional recombinant methods, isolation from HEC, etc.

For preparation of polyclonal antibodies, the first step is immunization of the host animal with the target protein, where the target protein will preferably be in substantially pure form, comprising less than about 1% contaminant. The immunogen may include the complete target protein, fragments or derivatives thereof. To increase the immune response of the host animal, the target protein may be combined with an adjuvant, where suitable adjuvants include alum, dextran, sulfate, large polymeric anions, oil & water emulsions, e.g. Freund's adjuvant, Freund's complete adjuvant, and the like. The target protein may also be conjugated to synthetic carrier proteins or synthetic antigens. A variety of hosts may be immunized to produce the polyclonal antibodies. Such hosts include rabbits, guinea pigs, rodents, e.g. mice, rats, sheep, goats, and the like. The target protein is administered to the host, usually intradermally, with an initial dosage followed by one or more, usually at least two, additional booster dosages. Following immunization, the blood from the host will be collected, followed by separation of the serum from the blood cells. The Ig present in the resultant antiserum may be further fractionated using known methods, such as ammonium salt fractionation, DEAE chromatography, and the like.

Monoclonal antibodies of the subject invention may be produced by conventional techniques. Generally, the spleen and/or lymph nodes of an immunized host animal provide a source of plasma cells. The plasma cells are immortalized by fusion with myeloma cells to produce hybridoma cells. Culture supernatant from individual hybridomas is screened using standard techniques to identify those producing antibodies with the desired specificity. Suitable animals for production of monoclonal antibodies to the human protein include mouse, rat, hamster, etc. To raise antibodies against the mouse protein, the animal will generally be a hamster, guinea pig, rabbit, etc. The antibody may be purified from the hybridoma cell supernatants or ascites fluid by conventional techniques, e.g. affinity chromatography using MPTS bound to an insoluble support, protein A sepharose, etc.

The antibody may be produced as a single chain, instead of the normal multimeric structure. Single chain antibodies are described in Jost et al. (1994) J.B.C. 269:26267-73, and others. DNA sequences encoding the variable region of the heavy chain and the variable region of the light chain are ligated to a spacer encoding at least about 4 amino acids of small neutral amino acids, including glycine and/or serine. The protein encoded by this fusion allows assembly of a functional variable region that retains the specificity and affinity of the original antibody.

### Generation of Antibodies

Also provided are methods of generating antibodies, e.g., monoclonal antibodies. In one embodiment, the blocking or inhibition, either directly or indirectly as described above, of the Site C repressor site/Site C repressor protein complex interaction is used to immortalize cells in culture, e.g., by enhancing telomerase expression. Exemplary of cells that may be used for this purpose are non-transformed antibody producing cells, e.g. B cells and plasma cells which may be isolated and identified for their ability to produce a desired antibody using known technology as, for example, taught in U.S. Pat. No. 5,627,052. These cells may either secrete antibodies (antibody-secreting cells) or maintain antibodies on the surface of the cell without secretion into the cellular environment. Such cells have a limited lifespan in culture, and are usefully immortalized by upregulating expression of telomerase using the methods of the present invention.

Because the above-described methods are methods of increasing expression of TERT and therefore increasing the proliferative capacity and/or delaying the onset of senescence in a cell, they find applications in the production of a range of reagents, typically cellular or animal reagents. For example, the subject methods may be employed to increase proliferation capacity, delay senescence and/or extend the lifetimes of cultured cells. Cultured cell populations having enhanced TERT expression are produced using any of the protocols as described above.

The subject methods find use in the generation of monoclonal antibodies. An antibody-forming cell may be identified among antibody-forming cells obtained from an animal which has either been immunized with a selected substance, or which has developed an immune response to an antigen as a result of disease. Animals may be immunized with a selected antigen using any of the techniques well known in the art suitable for generating an immune response. Antigens may include any substance to which an antibody may be made, including, among others, proteins, carbohydrates, inorganic or organic molecules, and transition state analogs that resemble intermediates in an enzymatic process. Suitable antigens include, among others, biologically active proteins, hormones, cytokines, and their cell surface receptors, bacterial or parasitic cell membrane or purified components thereof, and viral antigens.

As will be appreciated by one of ordinary skill in the art, antigens which are of low immunogenicity may be accompanied with an adjuvant or hapten in order to increase the immune response (for example, complete or incomplete Freund's adjuvant) or with a carrier such as keyhole limpet hemocyanin (KLH).

Procedures for immunizing animals are well known in the art. Briefly, animals are injected with the selected antigen against which it is desired to raise antibodies. The selected antigen may be accompanied by an adjuvant or hapten, as discussed above, in order to further increase the immune response. Usually the substance is injected into the peritoneal cavity, beneath the skin, or into the muscles or bloodstream. The injection is repeated at varying intervals and the immune response is usually monitored by detecting antibodies in the serum using an appropriate assay that detects the properties of the desired antibody. Large numbers of antibody-forming cells can be found in the spleen and lymph node of the immunized animal. Thus, once an immune response has been generated, the animal is sacrificed, the spleen and lymph nodes are removed, and a single cell suspension is prepared using techniques well known in the art.

Antibody-forming cells may also be obtained from a subject which has generated the cells during the course of a selected disease. For instance, antibody-forming cells from a human with a disease of unknown cause, such as rheumatoid arthritis, may be obtained and used in an effort to identify antibodies which have an effect on the disease process or which may lead to identification of an etiological agent or body component that is involved in the cause of the disease. Similarly, antibody-forming cells may be obtained from subjects with disease due to known etiological agents such as malaria or AIDS. These antibody forming cells may be derived from the blood or lymph nodes, as well as from other diseased or normal tissues. Antibody-forming cells may be prepared from blood collected with an anticoagulant such as heparin or EDTA. The antibody-forming cells may be further separated from erythrocytes and polymorphs using standard procedures such as centrifugation with Ficoll-Hypaque (Pharmacia, Uppsula, Sweden). Antibody-forming cells may also be prepared from solid tissues such as lymph nodes or tumors by dissociation with enzymes such as collagenase and trypsin in the presence of EDTA.

Antibody-forming cells may also be obtained by culture techniques such as in vitro immunization. Briefly, a source of antibody-forming cells, such as a suspension of spleen or lymph node cells, or peripheral blood mononuclear cells are cultured in medium such as RPMI 1640 with 10% fetal bovine serum and a source of the substance against which it is desired to develop antibodies. This medium may be additionally supplemented with amounts of substances known to enhance antibody-forming cell activation and proliferation such as lipopolysaccharide or its derivatives or other bacterial adjuvants or cytokines such as IL-1, IL-2, IL-4, IL-5, IL-6, GM-CSF, and IFN-γ. To enhance immunogenicity, the selected antigen may be coupled to the surface of cells, for example., spleen cells, by conventional techniques such as the use of biotin/avidin as described below.

Antibody-forming cells may be enriched by methods based upon the size or density of the antibody-forming cells relative to other cells. Gradients of varying density of solutions of bovine serum albumin can also be used to separate cells according to density. The fraction that is most enriched for desired antibody-forming cells can be determined in a preliminary procedure using the appropriate indicator system in order to establish the antibody-forming cells.

The identification and culture of antibody producing cells of interest is followed by enhancement of TERT expression in these cells by the subject methods, thereby avoiding the need for the immortalization/fusing step employed in traditional hybridoma manufacture protocols. In such methods, the first step is immunization of the host animal with an immunogen, typically a polypeptide, where the polypeptide will preferably be in substantially pure form, comprising less than about 1% contaminant. The immunogen may comprise the complete protein, fragments or derivatives thereof. To increase the immune response of the host animal, the protein may be combined with an adjuvant, where suitable adjuvants include alum, dextran sulfate, large polymeric anions, oil & water emulsions, e.g. Freund's adjuvant, Freund's complete adjuvant, and the like. The protein may also be conjugated to synthetic carrier proteins or synthetic antigens. A variety of hosts may be immunized to produce the subject antibodies. Such hosts include rabbits, guinea pigs, rodents (e.g. mice, rats), sheep, goats, and the like. The protein is administered to the host, usually intradermally, with an initial dosage followed by one or more, usually at least two, additional booster dosages. Following immunization, generally, the spleen and/or lymph nodes of an immunized host animal provide a source of plasma cells. The plasma cells are treated according to the subject invention to enhance TERT expression and thereby, increase the proliferative capacity and/or delay senescence to produce “pseudo” immortalized cells. Culture supernatant from individual cells is then screened using standard techniques to identify those producing antibodies with the desired specificity. Suitable animals for production of monoclonal antibodies to a human protein include mouse, rat, hamster, etc. To raise antibodies against the mouse protein, the animal will generally be a hamster, guinea pig, rabbit, etc. The antibody may be purified from the cell supernatants or ascites fluid by conventional techniques, e.g. affinity chromatography using RFLAT-1 protein bound to an insoluble support, protein A sepharose, etc.

In an analogous fashion, the subject methods are employed to enhance TERT expression in non-human animals, e.g., non-human animals employed in laboratory research. Using the subject methods with such animals can provide a number of advantages, including extending the lifetime of difficult and/or expensive to produce transgenic animals. As with the above described cells and cultures thereof, the expression of TERT in the target animals may be enhanced using a number of different protocols, including the administration of an agent that inhibits Site C repressor protein repression and/or targeted disruption of the Site C repressor binding site. The subject methods may be used with a number of different types of animals, where animals of particular interest include mammals, e.g., rodents such as mice and rats, cats, dogs, sheep, rabbits, pigs, cows, horses, and non-human primates, e.g. monkeys, baboons, etc.

### Screening Assays

Also provided by the subject invention are screening protocols and assays for identifying agents that modulate, e.g., inhibit or enhance, Site C repression of TERT transcription. The screening methods include assays that provide for qualitative/quantitative measurements of TERT promoter controlled expression, e.g., of a coding sequence for a marker or reporter gene, in the presence of a particular candidate therapeutic agent. Assays of interest include assays that measures the TERT promoter controlled expression of a reporter gene (i.e. coding sequence, e.g., luciferase, SEAP, etc.) in the presence and absence of a candidate inhibitor agent, e.g., the expression of the reporter gene in the presence or absence of a candidate agent. The screening method may be an in vitro or in vivo format, where both formats are readily developed by those of skill in the art. Whether the format is in vivo or in vitro, an expression system, e.g., a plasmid, that includes a Site C repressor binding site, a TERT promoter and a reporter coding sequence all operably linked is combined with the candidate agent in an environment in which, in the absence of the candidate agent, the TERT promoter is repressed, e.g., in the presence of the Site C repressor protein complex that interacts with the Site C repressor binding site and causes TERT promoter repression. The conditions may be set up in vitro by combining the various required components in an aqueous medium, or the assay may be carried out in vivo, e.g., in a cell-that normally lacks-telomerase activity, e.g., an MRC5 cell, etc.

As such, the present invention also provides methods for screening potential therapeutic agents useful for regulating the activity of TERT, replicative capacity of cells, and for treating conditions associated with the activity of TERT. According to the present invention, any agent that specifically increases or decreases the activity of a Site C regulator with respect to 1) its interaction with one or more repressor sites in TERT promoter or 2) its impact on the activity of TERT is a potential therapeutic agent capable of regulating the activity of TERT. Such screening can be carried out either in vitro, e.g., via high throughput screening in a test tube or tissue culture or in vivo, e.g., in animal models. In one embodiment, agents are tested for their ability to specifically bind to or interact with a Site C regulator and any specific binding between an agent and a Site C regulator is indicative of the agent's ability to regulate the activity of TERT.

In vitro models of repressor protein function are provided. Of particular interest are models of repressor protein TERT binding events in which the TERT binding site is Site C. Such models typically include: a Site C site, a repressor protein polypeptide and a modulatory agent, e.g., competitor or inhibitor, which are present under conditions sufficient to inhibit repressor protein/site C binding. The competitor may be any compound that is, or is suspected to be, a compound capable of specifically binding to the repressor protein, where of particular interest in many embodiments is the use of the subject ligands described above as competitors. Depending on the particular model, one or more of, usually one of, the specified components may be labeled, where by labeled is meant that the components comprise a detectable moiety, e.g. a fluorescent or radioactive tag, or a member of a signal producing system, e.g. biotin for binding to an enzyme-streptavidin conjugate in which the enzyme is capable of converting a substrate to a chromogenic product.

The above in vitro models may be designed in a number of different ways, where a variety of assay configurations and protocols may be employed, as are known in the art. For example, one of the components may be bound to a solid support, and the remaining components contacted with the support bound component. The above components of the method may be combined at substantially the same time or at different times, e.g. soluble repressor protein and a competitor ligand may be combined first, and the resultant mixture subsequently combined with bound site C sequence. Following the contact step, the subject methods will generally, though not necessarily, further include a washing step to remove unbound components, where such a washing step is generally employed when required to remove label that would give rise to a background signal during detection, such as radioactive or fluorescently labeled non-specifically bound components. Following the optional washing step, the presence of bound repressor protein/Site C complexes will then be detected.

In alternative in vitro models, an expression cassette including a reporter gene under control of a Site C sequence and repressor-protein may be present in a cell free environment in which the reporter gene is expressed in the absence of repressor protein binding to the Site C region. By expression cassette or system is meant a nucleic acid that includes a sequence encoding a peptide or protein of interest, i.e., a coding sequence, operably linked to a promoter sequence, where by operably linked is meant that expression of the coding sequence is under the control of the promoter sequence. The expression systems and cassettes of the subject invention include a Site C repressor binding site/region operably linked to the promoter, where the promoter is, in many embodiments, a TERT promoter, such as the hTERT promoter. See e.g., the hTERT promoter sequence described in Cong et al., Hum. Mol. Genet. (1999) 8:137-142. The in vitro model further includes a coding sequence of interest operably linked to the Site C binding site. The expression system is then employed in an appropriate cell free environment that includes the repressor protein to provide expression or non-expression of the protein, as desired.

A variety of different in vivo models of repressor protein function are also provided by the subject invention and may be used in the screening assays of the subject invention. In vivo models of interest include engineered cells that include an expression cassette as described above and a repressor protein, which components are present in a host cell. Also of interest in the subject screening assays are multicellular in vivo models, e.g., the transgenic animal models described below.

Whether the format is in vivo or in vitro, the model being employed is combined with the candidate agent and the effect of the candidate agent on model is observed and related to the TERT expression modulatory activity of the agent. For example, for screening inhibitory agents, the-model is combined with the candidate agent in an environment in which, in the absence of the candidate agent, the TERT promoter is repressed, e.g., in the presence of a repressor protein, that interacts with the TERT Site C repressor binding site and causes TERT promoter repression. The conditions may be set up in vitro by combining the various required components in an aqueous medium, or the assay may be carried out in vivo, etc.

Alternatively, the repressor protein could be engineered to replace the repressor domain with an activation domain (or other detectable domain), but still retaining the DNA binding domain. In this manner, assays can be set up in which agents that are candidates for preventing the repressor protein DNA binding domain from binding to the DNA binding site can be screened (as described in the above paragraph) for activation (or other signal) of the reporter gene instead of repression. Likewise, the repressor protein could be engineered to replace the DNA binding domain with another DNA binding domain (e.g. p53), but still retaining the repression domain. In this manner, assays can be set up in which agents that are candidates for preventing the repression domain from binding to cofactors (protein-protein interaction) can be screened using DNA binding domains that have already been well characterized. In this manner, agents that enhance and inhibit protein-protein interactions with cofactors involved in TERT expression repression may be identified.

A variety of different candidate agents may be screened by the above methods. Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Small molecule agents of particular interest include pyrrole-imidazole polyamides, analogous to those described in Dickinson et al., Biochemistry Aug. 17, 1999;38(33):10801-7. Other agents include “designer” DNA binding proteins that bind one or more repressor sites associated with Site C, e.g., without causing repression and prevent other Site C regulators from interacting with the repressor sites.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation,-alkylation, esterification, amidification, etc. to produce structural analogs.

Agents identified in the above screening assays that inhibit Site C repression of TERT transcription find use in the methods described above, e.g., in the enhancement of TERT expression. Alternatively, agents identified in the above screening assays that enhance Site C repression find use in applications where inhibition of TERT expression is desired, e.g., in the treatment of disease conditions characterized by the presence of unwanted TERT expression, such as cancer and other diseases characterized by the presence of unwanted cellular proliferation, where such methods are described in, for example, U.S. Pat. Nos. 5,645,986; 5,656,638; 5,703,116; 5,760,062; 5,767,278; 5,770,613; and 5,863,936; the disclosures of which are herein incorporated by reference.

The following examples are offered by way of illustration and not by way of limitation.

### Experimental

### EXAMPLE 1

### Purification of Site C Protein

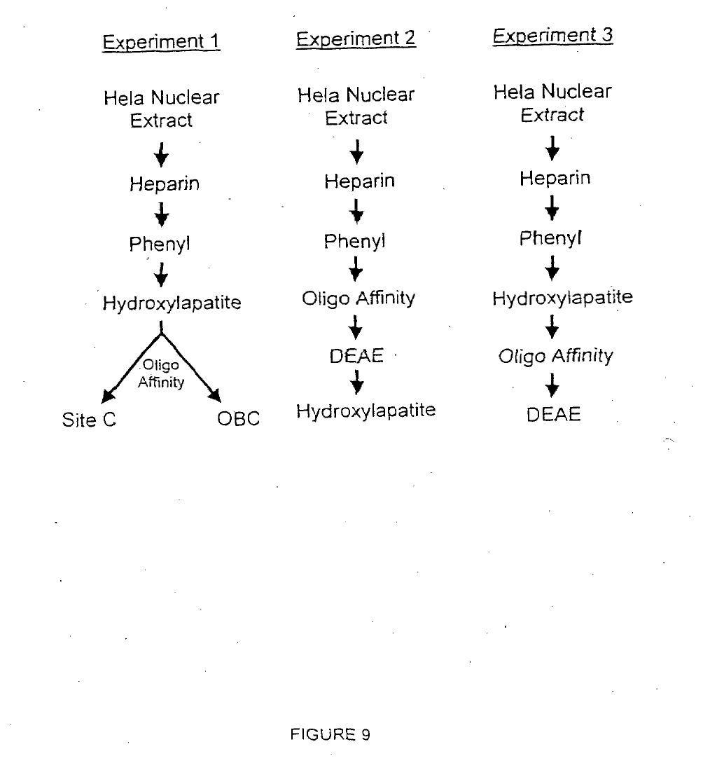

We have purified a group of Site C proteins from Hela nuclear cell extract using heparin chromatography, phenyl chromatography, hydroxylapatite chromatography, oligo affinity chromatography, and DEAE chromatography. The general schemes of the purification procedure is shown in FIG. 9. For the following set of experiments, one unit of activity means the amount of activity required to shift one fmole of Site C oligo in an electrophoretic mobility shift assay (EMSA) whereas X amount of specific activity means X fmoles of Site C binding activity per pg of protein. All fractions were assayed using Bradford assay to determine protein content. Specific activity was determined using EMSA.

### Experiment 1

### Heparin Column Chromatography

Nineteen heparin column chromatography runs were performed to process 1.49 liters of Hela nuclear extract having a specific activity of 0.59. All fractions having specific activities ranging from 1-10 were pooled to make pool B. Fractions having specific activities higher than 10 were pooled to make pool A.

The final pool B includes a total of 480 mls of fractions containing 1.49×10⁶ units of activity and 218 mg of protein for a specific activity of 6.8 (11.6 fold purification).

### Phenyl Column Chromatography

A total of 249 mls of pool B from heparin chromatography was run on phenyl column chromatography. All fractions having a specific activity greater than 40 were pooled. This phenyl pool had 47 mls in volume and contained 999,700 units of activity including 16.5 mg of protein for a specific activity of 60.6 (8.9 fold purification).

### Hydroxylapatite Column Chromatography

The entire 47 mls of phenyl pool was run on one hydroxylapatite column. Fractions having a specific activity greater than 50 were pooled to make HA pool. This pool had 10 mls in volume and contained 340,000 units of activity including 2.17 mg of protein for a specific activity of 156.7 (2.6 fold purification). This 10 ml pool was dialyzed in dialysis buffer containing 50 mM KCl. After dialysis the total volume-was reduced to 8.4 mls.

### Oligo Affinity Chromatography

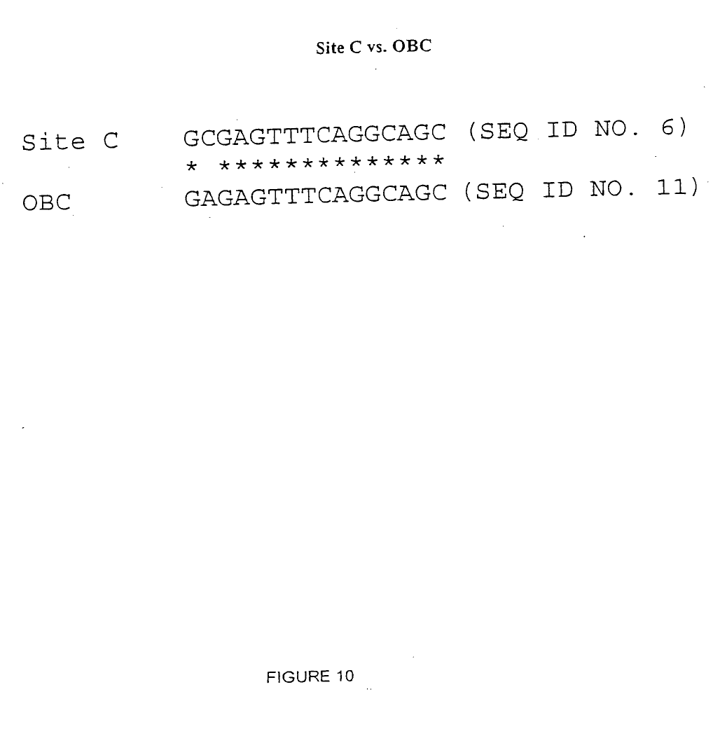

Column resins including biotinylated oligos conjugated to NeutrAvidin were prepared. Two columns were prepared that differed only in the sequence of the oligo that was used. One column contained the double stranded Site C oligo (TCGCGGCGCGAGTTTCAGGCAGCGCTGCGT, SEQ ID NO. 6) while the other column contained the double stranded OBC oligo (TCGCGGCGAGAGTTTCAGGCAGCGCTGCGT, SEQ ID NO. 11). The OBC oligo is identical to the Site C oligo except for one base as shown in FIG. 10. Twenty-five percent (25%) of the dialyzed HA pool was run on each column. One fraction off the Site C column contained 25,332 units of activity and 36.7 μg of protein for a specific activity of 690 (4.4 fold purification). The equivalent fraction from the OBC column contained four fold less activity (6,300 units) and 2.8 fold more protein (103 μg) for an −11.3 fold lower specific activity (61.2).

### SDS-PAGE

A total of 15 μl (380 units) of the peak fraction from the Site C column and 15 μl (95 units) of the equivalent fraction from the OBC column were run on an 8-16% SDS-PAGE gel and silver stained.

Proteins that specifically bind Site C should appear four times more abundant in the lane containing the Site C fraction, while proteins that do not specifically bind Site C should appear 2.8 times more abundant in the lane containing the OBC fraction. We had seen in silver stained SDS-PAGE gel several protein bands were more abundant in the OBC lane than in the Site C lane. However there were three bands, A, B, and C, that were more abundant in the Site C lane.

### Mass Spectrometry

This gel was sent to Charles River Proteomics who cut out bands A and B from the gel and identified them by Mass Spect (according to the protocol described in Journal of Proteome Research 3:303-311, 2003). One band was identified as human LBP-1b and one band was identified as human LBP1c (also called LSF). These proteins are more than 70% identical though they are encoded by separate genes on separate chromosomes. The amino acid and encoding nucleotide sequences for LBP-1b can be found in Genbank under the accession No. AAB29977. The amino acid and encoding nucleotide sequences for LBP-1c (LSF) can be found in Genbank under the accession No. NP_{—}005644 also AAB29976.

### Experiment 2

### Heparin Column Chromatography

Twelve heparin column chromatography runs were performed to process 1.35 liters of Hela nuclear extract and 147.5 ml heparin pool A from experiment 1. Column fractions having specific activities ranging from 16.67-38.893 were pooled to make a heparin pool C of 145 mls in volume containing 3.34×10⁶ units of activity and 142 mg protein for a specific activity of 23.54 (33.6 fold purification).

A separate heparin pool D was made using certain column fractions from the heparin chromatography. This pool had 21.5 mls in volume containing 18 units of activity and 28 mg protein for a specific activity of 15.6 (26.36 fold purification).

A total of 61.5 mls of heparin pool C and 21.5 mls of heparin pool D were combined to make a heparin pool E of 83 mls in volume containing 1.87×10⁶ units of activity and 89.1 mg protein for a specific activity of 20.95 (30 fold purification).

### Phenyl Column Chromatography

A total of 83 mls of heparin pool E was run on phenyl column chromatography. Column fractions 24-31 were pooled and dialyzed in dialysis buffer containing 50 mM KCL which resulted in a phenyl pool of 11 mls in volume containing 509,605 units of activity and 13.4 mg protein for a specific activity of 37.89 (1.8 fold purification).

### Oligo Affinity Chromatography

Column resins including biotinylated oligos conjugated to NeutrAvidin were prepared. The entire 11 mls of phenyl pool was run on the Site C oligo affinity column.

Two pools were made from the oligo affinity columns: one from fractions 18-25 (oligo affinity chromatography pool 1) and one from fractions 54-56 (oligo affinity chromatography pool 2). Pool 1 had 7.97 mls in volume containing 60,903 units of activity and 1.22 mg protein for a specific activity of 49.9 (1.3 fold purification). Pool 2 had 3 mls in volume containing 52,845 units of activity and 1.29 mg protein for a specific activity of 41.5 (1.09 fold purification).

### DEAE Column Chromatography

Both oligo affinity chromatography pools were run on one DEAE column. Fractions 29-32 were pooled to make a DEAE pool of 2 mls with 11,165 units of activity and 0.047 mg of protein for a specific activity of 236.3 (9.51 fold purification).

### Hydroxylapatite Column Chromatography

The entire 2 mls of DEAE pool was run on one hydroxylapatite column. Fractions 19 to 20 were pooled for a total volume of 2.94 mls with 3045 units of activity and undetectable amounts of protein by Bradford and 0.075 mg protein as measured using mAU for a specific activity of 40.4 (4.2 fold purification).

### SDS-PAGE

A total of 2.83 ml of the hydroxylapatite pool was concentrated to 50 μl. A total of 23 μl (437 units) of the concentrated pool was run on an 18% SDS-PAGE gel and a 7.5% SDS-PAGE gel. The gels were stained with SYPRO ruby.

### Mass Spectrometry

The two gels were sent to Charles River Proteomics where the bands were cut out from the gels and identified by Mass Spectrometry.

One band was identified as human CA150 (Sune et al., Mol. Cell. Biol. 17: 6029-6039 (1997). The amino acid and encoding nucleotide sequences for CA150 can be found in Genbank under the accession number AF017789. CA150 binding partners identified in the literature include Huntingtin (Holbert et al., PNAS USA 98: 1811-1816) and others.

Another band was identified as LBP1b (Huang et al., submitted (October 1999) to the EMBL/GenBank/DDBJ database; and Yoon et al., Mol. Cell. Biol. 14: 1776-1785 (1994). LBP-1b and LBP-1c are more than 70% identical though they are encoded by separate genes on separate chromosomes. The amino acid and encoding nucleotide sequences for LBP-1d can be found in Genbank under the accession number AF198487 and accession number AAB29977. The amino acid and encoding nucleotide sequences for LBP-1c (LSF) can be found in Genbank under the accession number BC003634 also NP_{—}005644 and AAB29976.

The third band was identified as Ras GTPase-activating-like protein IQGAP1 (Weissbach et al., J. boil. Chem. 269:20517-20521 (1994). Functions of IQGAP1 include binding to activated CDC42, however IQGAP1 does not stimulate CDC42's GTPase activity. In addition, IQGAP1 is associated with calmodulin and can serve as an assembly scaffold for the organization of a multimolecular complex that can interface incoming signals to the reorganization of the actin cytoskeleton at the plasma membrane. Tissue specificity of IQGAP1 includes expression in the placenta, lung, and kidney. A lower level expression is seen in the heart, liver, skeletal muscle and pancreas. The amino acid and encoding nucleotide sequences for IQGAP1 can be found in Genbank under the accession number L33075.

### Experiment 3

### Heparin Column Chromatography

One heparin column chromatography run was performed to process 274.76 mls of Hela nuclear extract. Fractions 38-43 were pooled to make a heparin pool of 84 mls in volume containing 982,777 units of activity and 77.197 mg proteins for a specific activity of 12.73 (25.75 fold purification).

### Phenyl Column Chromatography

A total of 83 mls of heparin pool was run on phenyl column chromatography. The phenyl pool was made from fractions 20-31 and had 24 mls in volume containing 809,829 units of activity and 9.34 mg protein for a specific activity of 86.7 (9.9 fold purification).

### Hydroxylapatite Column Chromatography

The entire 24 mls of phenyl pool was run on one hydroxylapatite column. Fractions 29-41 were pooled and dialyzed in dialysis buffer containing 50 mM KCl which resulted in a hydroxylapatite pool of 19.5 mls with 145,352 units of activity and 2.5 mg protein for a specific activity of 57.4 (1.7 fold purification).

### Oligo Affinity Chromatography

Column resins including biotinylated oligos conjugated to NeutrAvidin were prepared. The entire 19.5 mls of hydroxylapaptite pool was run on the Site C oligo affinity column with a triple repeat double strand Site C oligo (TCGCGGCGCGAGTTTCAGGCAGCGCTGGCGCGAGTTTCAGGCAGCGCTGGCG CGAGTTTCAGGCAGCGCTGCGT, SEQ ID NO. 12). Fractions 30-33 were pooled to make an oligo affinity pool of 4 mls with 85,562 units of activity and 0.24 mg protein for a specific activity of 343.3 (4.65 fold purification).

### DEAE Column Chromatography

The oligo affinity pool was run on one DEAE column. Fractions 18-24 were pooled and dialyzed to make a DEAE pool of 1.4 mls with 61,475 units of activity and 0.108 mg protein for a specific activity of 566.6 (1.46 fold purification).

### SDS-PAGE

A total of 9.11 Tl, representing 400 units of activity from the DEAE pool was run on a 4-12% XT Criterion PAGE gel and stained with SYPRO ruby. This gel was labeled Project 625a. A total of 22.7 Tl, representing 1000 units of activity from the DEAE pool was run on a 4-12% XT Criterion PAGE gel and stained with SYPRO ruby. This gel was labeled Project 625 g.

### Mass Spectrometry

A 360 μl aliquot of the DEAE pool, containing 15,000 units of activity was sent to Charles River Proteomics where it was analyzed by LC/MS/MS. Two proteins were identified, LBP1c and LBP9. A small portion of undigested sample was also analyzed using the linear mode on the MALDI-TOF Mass Spec where two protein species were identified, one with an estimated size of 60 kilo-daltons and one with an estimated size of 53 kilo-daltons.

Four protein bands separated from the DEAE pool on the Project 652 g gel were cut out and labeled bands A-D. These bands were sent to the Proteomics Core Facility at the University of Nevada, Reno where they were identified by MALDI-TOF Mass Spectrometry. Band A was identified as LBP1b and/or LBP1a. Bands B and C were identified as transcription factor CP2, and/or LBP1d and/or LSF.

### Features of the Invention

Accordingly the present invention provides compositions and methods useful for regulating TERT activity and treating conditions associated with TERT activity. In addition, the present invention provides methods for screening potential therapeutic agents useful for regulating TERT activity.

In one embodiment, the present invention provides a method of modulating the expression of telomerase reverse transcriptase in a cell. The method includes contacting the cell with a Site C regulator, wherein the Site C regulator modulates the activity of Site C whereby modulating the expression of telomerase reverse transcriptase.

In another embodiment, the present invention provides a method of treating a condition in a subject. The method includes administering to a subject an effective amount of a Site C regulator, wherein the condition is associated with telomerase reverse transcriptase activity and wherein the Site C regulator modulates the activity of Site C whereby modulating the expression of telomerase reverse transcriptase.

In yet another embodiment, the present invention provides a method of regulating the replicative capacity of a cell. The method includes contacting the cell with a Site C regulator, wherein the Site C regulator modulates the activity of Site C whereby modulating the replicative capacity of the cell.

In yet another embodiment, the present invention provides a method of screening potential therapeutic agents for the ability to regulate the expression of telomerase reverse transcriptase. The method includes contacting a potential therapeutic agent with a Site C regulator, wherein an increase or decrease of activity of the Site C regulator caused by the potential therapeutic agent indicates that the agent is capable of regulating the expression of telomerase reverse transcriptase.

In still another embodiment, the present invention provides a method of screening potential therapeutic agents for the ability to regulate the expression of telomerase reverse transcriptase. The method includes contacting a potential therapeutic agent with a Site C regulator, wherein a potential therapeutic agent specifically binding to the Site C regulator is an agent capable of regulating the expression of telomerase reverse transcriptase.

In another embodiment, the present invention provides a method of interacting with Site C in a cell. The method includes contacting the cell with a Site C protein that is a member of LSF family.

In yet another embodiment, the present invention provides a method of interacting with Site C in a cell. The method includes contacting the cell with a Site C protein selected from the group consisting of HKR3, ZNF140, ZFP161, Solute Carrier Family 3, Splicing Factor 3A, Ran-GTP, ELG, BCL6, Matrin3, BMAL2, U2 snRNA Protein, LZ16, PC4, F13, TCFL5, p65, c-Rel, Proteosome, p42POP, NF45, CA150, MRG15, ZNF135, Ras GTPase, PHD7, WBSCR2, E2F3B, E2F3, p107, Huntingtin, p231HBP, DP1, DP2, YY1, NF-E4, Fe65, APP-CT, NFPB, SP1, SP2, SP3, SP4, TIEG1, TIEG2, BTEB1, BTEB2, BTEB3, ZF9, ZNF741, UKLF, BKLF, IKLF, GKLF, LKLF, EKLF, AP-2rep, TFIIB, TBP, TAF55, TAF135, CRSP, RB, p53, HCF1, KIAA0461, Dorfin, Atf7ip, E2F, Oct1, GATA1, RelA, TIEG, ELF1, SREBP2, Hsc70, SF3A120, HSph2, and KIAA1903.

In another embodiment, the present invention provides a polypeptide comprising an amino acid sequence of LBP1c2 as shown in SEQ ID NO. 01 and a polynucleotide comprising a nucleic acid sequence encoding LBP1c2 as shown in SEQ ID NO. 02.

(SEQ ID NO:02)

ATGGCCTGGG CTCTGAAGCT GCCTCTGGCC GACGAAGTGA
TTGAATCCGG GTTGGTGCAG TACCGGACCC GAGACTTCGA
CGGAGACCGG CTGCTTCACT AACTTAGGCC CAACCACGTC
GACTTTGATG CTAGCCTGTC CGGGATCGGC CAGGAACTGG
GTGCTGGTGC CTATAGCATG CTGAAACTAC GATCGGACAG
GCCCTAGCCG GTCCTTGACC CACGACCACG GATATCGTAC
AGCACCTTTT CATATGTTTG TGGGCCACTG ATGTTTTCTC
CTAAGAATGA TGTCCTTGCA TCGTGGAAAA GTATACAAAC
ACCCGGTGAC TACAAAAGAG GATTCTTACT ACAGGAACGT
TTGCCCATTT TTAAGCAAGA AGAGTCGAGT TTGCCTCCTG
ATAATGAGAA TAAAATCCTG AACGGGTAAA AATTCGTTCT
TCTCAGCTCA AACGGAGGAC TATTACTCTT ATTTTAGGAC
CCTTTTCAAT ATGTGCTTTG TGCTGCTACC TCTCCAGCAG
TGAAACTCCA TGATGAAACC GGAAAACTTA TACACGAAAC
ACGACGATGG AGAGGTCGTC ACTTTGAGGT ACTACTTTGG
CTAACGTATC TCAATCAAGG ACAGTCTTAT GAAATTCGAA
TGCTAGACAA TAGGAAACTT GATTGCATAG AGTTAGTTCC
TGTCAGAATA CTTTAAGCTT ACGATCTGTT ATCCTTTGAA
GGAGAACTTC CAGAAATTAA TGGCAAATTG GTGAAGAGTA
TATTCCGTGT GGTGTTCCAT CCTCTTGAAG GTCTTTAATT
ACCGTTTAAC CACTTCTCAT ATAAGGCACA CCACAAGGTA
GACAGAAGGC TTCAGTACAC TGAGCATCAG CAGCTAGAGG
GCTGGAGGTG GAACCGACCT CTGTCTTCCG AAGTCATGTG
ACTCGTAGTC GTCGATCTCC CGACCTCCAC CTTGGCTGGA
GGAGACAGAA TTCTTGACAT AGATATCCCG ATGTCTGTGG
GTATAATCGA TCCTAGGGCT CCTCTGTCTT AAGAACTGTA
TCTATAGGGC TACAGACACC CATATTAGCT AGGATCCCGA
AATCCAACTC AACTAAATAC AGTGGAGTTC CTGTGGGACC
CTGCAAAGAG GACATCTGTG TTAGGTTGAG TTGATTTATG
TCACCTCAAG GACACCCTGG GACGTTTCTC CTGTAGACAC
TTTATTCAGG TGCACTGTAT TAGCACAGAG TTCACTATGA
GGAAACATGG TGGAGAAAAG AAATAAGTCC ACGTGACATA
ATCGTGTCTC AAGTGATACT CCTTTGTACC ACCTCTTTTC
GGGGTGCCAT TCCGAGTACA AATAGATACC TTCAAGGAGA
ATGAAAACGG GGAATATACT CCCCACGGTA AGGCTCATGT
TTATCTATGG AAGTTCCTCT TACTTTTGCC CCTTATATGA
GAGCACTTAC ACTCGGCCAG CTGCCAGATC AAAGTTTTCA
AGCCCAAAGG TGCAGACAGA CTCGTGAATG TGAGCCGGTC
GACGGTCTAG TTTCAAAAGT TCGGGTTTCC ACGTCTGTCT
AAGCAAAAAA CGGATAGGGA AAAAATGGAG AAACGAACAC
CTCATGAAAA GGAGAAATAT TTCGTTTTTT GCCTATCCCT
TTTTTACCTC TTTGCTTGTG GAGTACTTTT CCTCTTTATA
CAGCCTTCCT ATGAGACAAC CATACTCACA GAGTGTTCTC
CATGGCCCGA GATCACGTAT GTCGGAAGGA TACTCTGTTG
GTATGAGTGT CTCACAAGAG GTACCGGGCT CTAGTGCATA
GTCAATAACT CCCCATCACC TGGCTTCAAC AGTTCCCATA
GCAGTTTTTC TCTTGGGGAA CAGTTATTGA GGGGTAGTGG
ACCGAAGTTG TCAAGGGTAT CGTCAAAAAG AGAACCCCTT
GGGATGGTGC GTCCAAGGTT AACCATTTAT GTTTGTCAGG
AATCACTGCA GTTGAGGGAG CCCTACCACG CAGGTTCCAA
TTGGTAAATA CAAACAGTCC TTAGTGACGT CAACTCCCTC
CAGCAACAAC AGCAGCAGCA ACACCAGGAG AAGCATGAGG
ATGGAGACTC AAATGGTACT GTCGTTGTTG TCGTCGTCGT
TGTCGTCGTC TTCGTACTCC TACCTCTGAG TTTACCATGA
TTCTTCGTTT ACCATGCTAT CTATCTAGAA GAACTAACAG
CTGTTGAATT GACAGAAAAA AAGAAGCAAA TGGTACGATA
GATACATCTT CTTGATTGTC GACAACTTAA CTGTCTTTTT
ATTGCTCAGC TTTTCAGCAT TTCCCCTTGC CAGATCAGCC
AGATTTACAA GCAGGGGCCA TAACGAGTCG AAAAGTCGTA
AAGGGGAACG GTCTAGTCGG TCTAAATGTT CGTCCCCGGT
ACAGGAATTC ATGTGCTCAT CAGTGATGAG ATGATACAGA
ACTTTCAGGA AGAAGCATGT TGTCCTTAAG TACACGAGTA
GTCACTACTC TACTATGTCT TGAAAGTCCT TCTTCGTACA
TTTATTCTGG ACACAATGAA AGCAGAAACC AATGATAGCT
ATCATATCAT ACTGAAG AAATAAGACC TGTGTTACTT
TCGTCTTTGG TTACTATCCA TAGTATAGTA TGACTTC

In another embodiment, the present invention provides a polypeptide comprising a LBP1c2 amino acid sequence as shown in SEQ ID NO. 03.

(SEQ ID NO:03)

MAWALKLPLA DEVIESGLVQ DFDASLSGIG QELGAGAYSM
STFSYVCGPL MFSPKNDVLA LPIFKQEESS LPPDNENKIL
PFQYVLCAAT SPAVKLHDET LTYLNQGQSY EIRMLDNRKL
GELPEINGKL VKSIFRVVFH DRRLQYTEHQ QLEGWRWNRP
GDRILDIDIP MSVGITDPRA NPTQLNTVEF LWDPAKRTSV
FIQVHCISTE FTMRKHGGEK GVPFRVQIDT FKENENGEYT
EHLHSASCQI KVFKPKGADR KQKTDREKME KRTPHEKEKY
QPSYETTILT ECSPWPEITY VNNSPSPGFN SSHSSFSLGE
GMVRPRLTIY VCQESLQLRE QQQQQQQQQQ KHEDGDSNGT
FFVYHAIYLE ELTAVELTEK IAQLFSISPC QISQIYKQGP
TGIHVLISDE MIQNFQEEAC FILDTMKAET NDSYHIILK

In yet another embodiment, the present invention provides a polypeptide comprising an amino acid sequence of BOMv2 as shown in SEQ ID NO. 04 and a polynucleotide comprising a nucleic acid sequence of BOMv2 as shown in SEQ ID NO. 05.

(SEQ ID NO:04)

MSQESDNNKR LVALVPMPSD PPFNTRRAYT SEDEAWKSYL
ENPLTAATKA MMSINGDEDS AAALGLLYDY YKVPRDKRLL
SVSKASDSQE DQEKRNCLGT SEAQSNLSGG ENRVQVLKTV
PVNLSLNQDH LENSKREQYS ISFPESSAII PVSGITVVKA
EDFTPVFMAP PVHYPRGDGE EQRVVIFEQT QYDVPSLATH
SAYLKDDQRS TPDSTYSESF KDAATEKFRS ASVGAEEYMY
DQTSSGTFQY TLEATKSLRQ KQGEGPMTYL NKGQFYAITL
SETGDNKCFR HPISKVRSVV MVVFSEDKKQ R

(SEQ ID NO:05)

ATGTCACAAG AGTCGGACAA TAATAGAAAA CTAGTGGCCT
TAGTGCCCAT GCCCAGTGAC TACAGTGTTC TCAGCCTGTT
ATTATCTTTT GATCACCGGA ATCACGGGTA CGGGTCACTG
CCTCCATTCA ATACCCGAAG AGCCTACACC AGTGAGGATG
AAGCCTGGAA GTCATACTTG GGAGGTAAGT TATGGGCTTC
TCGGATGTGG TCACTCCTAC TTCCGACCTT CAGTATGAAC
GAGAATCCCC TGACAGCAGC CACCAAGGCC ATGATGAGCA
TTAATGGTGA TGAGGACAGT CTCTTAGGGG ACTGTCGTCG
GTGCTTCCGG TACTACTCGT AATTACCACT ACTCCTGTCA
GCTGCTGCCC TCGGCCTGCT CTATGACTAC TACAAGGTTC
CACGAGACAA GAGGCTGCTG CGACGACGGG AGCCGGACGA
GATACTGATG ATGTTCCAAG GTGCTCTGTT CTCCGACGAC
TCTGTAAGCA AAGCAAGTGA CAGCCAAGAA GACCAGGAGA
AAAGAAACTG CCTTGGCACC AGACATTCGT TTCGTTCACT
GTCGGTTCTT CTGGTCCTCT TTTCTTTGAC GGAACCGTGG
AGTGAAGCCC AGAGTAATTT GAGTGGAGGA GAAAACCGAG
TGCAAGTCCT AAAGACTGTT TCACTTCGGG TCTCATTAAA
CTCACCTCCT CTTTTGGCTC ACGTTCAGGA TTTCTGACAA
CCAGTGAACC TTTCCCTAAA TCAAGATCAC CTGGAGAACT
CCAAGCGGGA ACAGTACAGC GGTCACTTGG AAAGGGATTT
AGTTCTAGTG GACCTCTTGA GGTTCGCCCT TGTCATGTCG
ATAAGCTTCC CCGAGAGCTC TGCCATCATC CCGGTGTCGG
GAATCACGGT GGTGAAAGCT TATTCGAAGG GGCTCTCGAG
ACGGTAGTAG GGCCACAGCC CTTAGTGCCA CCACTTTCGA
GAAGATTTCA CACCAGTTTT CATGGCCCCA CCTGTGCACT
ATCCCCGGGG AGATGGGGAA CTTCTAAAGT GTGGTCAAAA
GTACCGGGGT GGACACGTGA TAGGGGCCCC TCTACCCCTT
GAGCAACGAG TGGTTATCTT TGAACAGACT CAGTATGACG
TGCCCTCGCT GGCCACCCAC CTCGTTGCTC ACCAATAGAA
ACTTGTCTGA GTCATACTGC ACGGGAGCGA CCGGTGGGTG
AGCGCCTATC TCAAAGACGA CCAGCGCAGC ACTCCGGACA
GCACATACAG CGAGAGCTTC TCGCGGATAG AGTTTCTGCT
GGTCGCGTCG TGAGGCCTCT CGTGTATGTC GCTCTCGAAG
AAGGACGCAG CCACAGAGAA ATTTCGGAGT GCTTCAGTTG
GGGCTGAGGA GTACATGTAT TTCCTGCCTC GGTGTCTCTT
TAAAGCCTCA CGAAGTCAAC CCCGACTCCT CATGTACATA
GATCAGACAT CAAGTGGCAC ATTTCAGTAC ACCCTGGAAG
CCACCAAATC TCTCCGTCAG CTAGTCTGTA GTTCACCGTG
TAAAGTCATG TGGGACCTTC GGTGGTTTAG AGAGGCAGTC
AAGCAGGGGG AGGGCCCCAT GACCTACCTC AACAAAGGAC
AGTTCTATGC CATAACACTC TTCGTCCCCC TCCCGGGGTA
CTGGATGGAG TTGTTTCCTG TCAAGATACG GTATTGTGAG
AGCGAGACCG GAGACAACAA ATGCTTCCGA CACCCCATCA
GCAAAGTCAG GAGTGTGGTG TCGCTCTGGC CTCTGTTGTT
TACGAAGGCT GTGGGGTAGT CGTTTCAGTC CTCACACCAC
ATGGTGGTCT TCAGTGAAGA CAAAAAACAG AGATACCACC
AGAAGTCACT TCTGTTTTTT GTCTCT

In still another embodiment, the present invention provides a pharmaceutical composition useful for regulating telomerase reverse transcriptase. The composition includes a first Site C regulator and a second Site C regulator.

It is evident from the above results and discussion that the subject invention provides important methods and compositions that find use in a variety of applications, including the establishment of expression systems that exploit the regulatory mechanism of the TERT gene and the establishment of screening assays for agents that enhance TERT expression. In addition, the subject invention provides methods of enhancing TERT expression in a cellular or animal host, which methods find use in a variety of applications, including the production of scientific research reagents and therapeutic treatment applications. Accordingly, the subject invention represents significant contribution to the art.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. A method for modulating a binding event between Site C and a repressor protein complex made up of one or more proteins, said method comprising:
contacting said Site C and/or said repressor protein complex with a modulatory agent under conditions sufficient for binding between said Site C and repressor protein to be modulated, wherein said repressor protein complex includes a an LSF protein.

2. The method according to claim 1, wherein said method is a method of inhibiting binding between said Site C and said repressor protein complex.

3. The method according to claim 1, wherein said method is a method of enhancing binding between said Site C and said repressor protein complex.

4. The method according to claim 1, wherein said binding event is an in vitro binding event.

5. The method according to claim 1, wherein said binding event is an in vivo binding event.

6. The method according to claim 1, wherein said binding event occurs inside of a cell.

7. A method of modulating expression of TERT from a TERT expression system that includes a Site C binding site, said method comprising:
contacting said system with a modulatory agent under conditions sufficient for binding between said Site C and a Site C repressor protein complex made up of one or more proteins, wherein said repressor protein complex comprises an LSF protein.

8. The method according to claim 7, wherein said method is a method of inhibiting binding between said Site C and said repressor protein complex.

9. The method according to claim 7, wherein said method is a method of enhancing binding between said Site C and said repressor protein complex.

10. The method according to claim 7, wherein said binding event is an in vitro binding event.

11. The method according to claim 7, wherein said binding event is an in vivo binding event.

12. The method according to claim 7, wherein said binding event occurs inside of a cell.

13-38. (canceled)

39. A method of determining whether an agent reduces repression of TERT transcription by a Site C repressor protein complex made up of one or more proteins, said method comprising:
(a) contacting said agent with an expression system comprising a Site C sequence, said Site C repressor protein complex and a coding sequence under conditions such that in the absence of said agent, transcription of said coding sequence is repressed, wherein said repressor protein complex includes an LSF protein;
(b) determining whether transcription of said coding sequence is repressed in the presence of said agent; and
(c) identifying said agent as an agent that inhibits repression of TERT transcription if transcription of said coding sequence is not repressed in the presence of said agent.

40. The method according to claim 39, wherein said contacting step occurs in a cell-free environment.

41. The method according to claim 39, wherein said contacting step occurs in a cell.

42. The method according to claim 39, wherein said agent is a small molecule.

43. The method according to claim 39, wherein said cell is a human cell.

44-47. (canceled)


SEQUENCE LISTING (36 sequences)

SEQ ID NO: 1 - protein, 439 aa, human
MAWALKLPLADEVIESGLVQDFDASLSGIGQELGAGAYSMSTFSYVCGPLMFSPKNDVLALPIFKQEESSLPPDNENKILPFQYVLCAATSPAVKLHDETLTYLNQGQSYEIRMLDNRKLGELPEINGKLVKSIFRVVFHDRRLQYTEHQQLEGWRWNRPGDRILDIDIPMSVGIIDPRANPTQLNTVEFLWDPAKRTSVFIQVHCISTEFTMRKHGGEKGVPFRVQIDTFKENENGEYTEHLHSASCQIKVFKPKGADRKQKTDREKMEKRTPHEKEKYQPSYETTILTECSPWPEITYVNNSPSPGFNSSHSSFSLGEGMVRPRLTIYVCQESLQLREQQQQQQQQQQKHEDGDSNGTFFVYHAIYLEELTAVELTEKIAQLFSISPCQISQIYKQGPTGIHVLISDEMIQNFQEEACFILDTMKAETNDSYHIILK

SEQ ID NO: 2 - DNA, 2634 bp, human
atggcctgggctctgaagctgcctctggccgacgaagtgattgaatccgggttggtgcagtaccggacccgagacttcgacggagaccggctgcttcactaacttaggcccaaccacgtcgactttgatgctagcctgtccgggatcggccaggaactgggtgctggtgcctatagcatgctgaaactacgatcggacaggccctagccggtccttgacccacgaccacggatatcgtacagcaccttttcatatgtttgtgggccactgatgttttctcctaagaatgatgtccttgcatcgtggaaaagtatacaaacacccggtgactacaaaagaggattcttactacaggaacgtttgcccatttttaagcaagaagagtcgagtttgcctcctgataatgagaataaaatcctgaacgggtaaaaattcgttcttctcagctcaaacggaggactattactcttattttaggacccttttcaatatgtgctttgtgctgctacctctccagcagtgaaactccatgatgaaaccggaaaagttatacacgaaacacgacgatggagaggtcgtcactttgaggtactactttggctaacgtatctcaatcaaggacagtcttatgaaattcgaatgctagacaataggaaacttgattgcatagagttagttcctgtcagaatactttaagcttacgatctgttatcctttgaaggagaacttccagaaattaatggcaaattggtgaagagtatattccgtgtggtgttccatcctcttgaaggtctttaattaccgtttaaccacttctcatataaggcacaccacaaggtagacagaaggcttcagtacactgagcatcagcagctagagggctggaggtggaaccgacctctgtcttccgaagtcatgtgactcgtagtcgtcgatctcccgacctccaccttggctggaggagacagaattcttgacatagatatcccgatgtctgtgggtataatcgatcctagggctcctctgtcttaagaactgtatctatagggctacagacacccatattagctaggatcccgaaatccaactcaactaaatacagtggagttcctgtgggaccctgcaaagaggacatctgtgttaggttgagttgatttatgtcacctcaaggacaccctgggacgtttctcctgtagacactttattcaggtgcactgtattagcacagagttcactatgaggaaacatggtggagaaaagaaataagtccacgtgacataatcgtgtctcaagtgatactcctttgtaccacctcttttcggggtgccattccgagtacaaatagataccttcaaggagaatgaaaacggggaatatactccccacggtaaggctcatgtttatctatggaagttcctcttacttttgccccttatatgagagcacttacactcggccagctgccagatcaaagttttcaagcccaaaggtgcagacagactcgtgaatgtgagccggtcgacggtctagtttcaaaagttcgggtttccacgtctgtctaagcaaaaaacggatagggaaaaaatggagaaacgaacacctcatgaaaaggagaaatatttcgttttttgcctatcccttttttacctctttgcttgtggagtacttttcctctttatacagccttcctatgagacaaccatactcacagagtgttctccatggcccgagatcacgtatgtcggaaggatactctgttggtatgagtgtctcacaagaggtaccgggctctagtgcatagtcaataactccccatcacctggcttcaacagttcccatagcagtttttctcttggggaacagttattgaggggtagtggaccgaagttgtcaagggtatcgtcaaaaagagaaccccttgggatggtgcgtccaaggttaaccatttatgtttgtcaggaatcactgcagttgagggagccctaccacgcaggttccaattggtaaatacaaacagtccttagtgacgtcaactccctccagcaacaacagcagcagcaacagcagcagaagcatgaggatggagactcaaatggtactgtcgttgttgtcgtcgtcgttgtcgtcgtcttcgtactcctacctctgagtttaccatgattcttcgtttaccatgctatctatctagaagaactaacagctgttgaattgacagaaaaaaagaagcaaatggtacgatagatagatcttcttgattgtcgacaacttaactgtctttttattgctcagcttttcagcatttccccttgccagatcagccagatttacaagcaggggccataacgagtcgaaaagtcgtaaaggggaacggtctagtcggtctaaatgttcgtccccggtacaggaattcatgtgctcatcagtgatgagatgatacagaactttcaggaagaagcatgttgtccttaagtacacgagtagtcactactctactatgtcttgaaagtccttcttcgtacatttattctggacacaatgaaagcagaaaccaatgatagctatcatatcatactgaagaaataagacctgtgttactttcgtctttggttactatcgatagtatagtatgacttc

SEQ ID NO: 3 - protein, 439 aa, human; identical to SEQ ID NO: 1

SEQ ID NO: 4 - protein, 311 aa, human
MSQESDNNKRLVALVPMPSDPPFNTRRAYTSEDEAWKSYLENPLTAATKAMMSINGDEDSAAALGLLYDYYKVPRDKRLLSVSKASDSQEDQEKRNCLGTSEAQSNLSGGENRVQVLKTVPVNLSLNQDHLENSKREQYSISFPESSAIIPVSGITVVKAEDFTPVFMAPPVHYPRGDGEEQRVVIFEQTQYDVPSLATHSAYLKDDQRSTPDSTYSESFKDAATEKFRSASVGAEEYMYDQTSSGTFQYTLEATKSLRQKQGEGPMTYLNKGQFYAITLSETGDNKCFRHPISKVRSVVMVVFSEDKKQR

SEQ ID NO: 5 - DNA, 1866 bp, human
atgtcacaagagtcggacaataatagaaaactagtggccttagtgcccatgcccagtgactacagtgttctcagcctgttattatcttttgatcaccggaatcacgggtacgggtcactgcctccattcaatacccgaagagcctacaccagtgaggatgaagcctggaagtcatacttgggaggtaagttatgggcttctcggatgtggtcactcctacttcggaccttcagtatgaacgagaatcccctgacagcagccaccaaggccatgatgagcattaatggtgatgaggacagtctcttaggggactgtcgtcggtggttccggtactactcgtaattaccactactcctgtcagctgctgccctcggcctgctctatgactactacaaggttccacgagacaagaggctgctgcgacgacgggagccggacgagatactgatgatgttccaaggtgctctgttctccgacgactctgtaagcaaagcaagtgacagccaagaagaccaggagaaaagaaactgccttggcaccagacattcgtttcgttcactgtcggttcttctggtcctcttttctttgacggaaccgtggagtgaagcccagagtaatttgagtggaggagaaaaccgagtgcaagtcctaaagactgtttcacttcgggtctcattaaactcacctcctcttttggctcacgttcaggatttctgacaaccagtgaacctttccctaaatcaagatcacctggagaactccaagcgggaacagtacagcggtcacttggaaagggatttagttctagtggacctcttgaggttcgcccttgtcatgtcgataagcttccccgagagctctgccatcatcccggtgtcgggaatcacggtggtgaaagcttattcgaaggggctctcgagacggtagtagggccacagcccttagtgccaccactttcgagaagatttcacaccagttttcatggccccacctgtgcactatccccggggagatggggaacttctaaagtgtggtcaaaagtaccggggtggacacgtgataggggcccctctaccccttgagcaacgagtggttatctttgaacagactcagtatgacgtgccctcgctggccacccacctcgttgctcaccaatagaaacttgtctgagtcatactgcacgggagcgaccggtgggtgagcgcctatctcaaagacgaccagcgcagcactccggacagcacatacagcgagagcttctcgcggatagagtttctgctggtcgcgtcgtgaggcctgtcgtgtatgtcgctctcgaagaaggacgcagccacagagaaatttcggagtgcttcagttggggctgaggagtacatgtatttcctgcgtcggtgtctctttaaagcctcacgaagtcaaccccgactcctcatgtacatagatcagacatcaagtggcacatttcagtacaccctggaagccaccaaatctctccgtcagctagtctgtagttcaccgtgtaaagtcatgtgggaccttcggtggtttagagaggcagtcaagcagggggagggccccatgacctacctcaacaaaggacagttctatgccataacactcttcgtccccctcccggggtactggatggagttgtttcctgtcaagatacggtattgtgagagcgagaccggagacaacaaatgcttccgacaccccatcagcaaagtcaggagtgtggtgtcgctctggcctctgttgtttacgaaggctgtggggtagtcgtttcagtcctcacaccacatggtggtcttcagtgaagacaaaaaacagagataccaccagaagtcacttctgttttttgtctct

SEQ ID NO: 6 - DNA, 30 bp, human
tcgcggcgcgagtttcaggcagcgctgcgt

SEQ ID NO: 7 - DNA, 24 bp, human
cggcgcgagtttcaggcagcgctg

SEQ ID NO: 8 - DNA, 51 bp, human
gcgaggagagggcggggccgcggaatttttttccgcggccccgccctctcc

SEQ ID NO: 9 - DNA, 22 bp, human
tccgcggccccgccctctcctc

SEQ ID NO: 10 - DNA, 47 bp, human
tccgcggccccgccctctcctcgcggcgcgagtttcaggcagcgctg

SEQ ID NO: 11 - DNA, 30 bp, human
tcgcggcgagagtttcaggcagcgctgcgt

SEQ ID NO: 12 - DNA, 74 bp, human
tcgcggcgcgagtttcaggcagcgctggcgcgagtttcaggcagcgctggcgcgagtttcaggcagcgctgcgt

SEQ ID NO: 13 - DNA, 261 bp, human
ccaggaccgcgctccccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagtttcaggcagcgctgcgtcctgctgcgcacgtgggaagccctggccccggccacccccgcgatg

SEQ ID NO: 14 - protein, 501 aa, human
MAWALKLPLADEVIESGLVQDFDASLSGIGQELGAGAYSMSDVLALPIFKQEESSLPPDNENKILPFQYVLCAATSPAVKLHDETLTYLNQGQSYEIRMLDNRKLGELPEINGKLVKSIFRVVFHDRRLQYTEHQQLEGWRWNRPGDRILDIDIPMSVGIIDPRANPTQLNTVEFLWDPAKRTSVFIQVHCISTEFTMRKHGGEKGVPFRVQIDTFKENENGEYTEHLHSASCQIKVFKPKGADRKQKTDREKMEKRTPHEKEKYQPSYETTILTECSPWPEITYVNNSPSPGFNSSHSSFSLGEGNGSPNHQPEPPPPVTDNLLPTTTPQEAQQWLHRNRFSTFTRLFTNFSGADLLKLTRDDVIQICGPADGIRLFNALKGMVRPRLTIYVCQESLQLREQQQQQQQQQQKHEDGDSNGTFFVYHAIYLEELTAVELTEKIAQLFSISPCQISQIYKQGPTGIHVLISDEMIQNFQEEACFILDTMKAETNDSYHIILK

SEQ ID NO: 15 - protein, 627 aa, human
MSQESDNNKRLVALVPMPSDPPFNTRRAYTSEDEAWKSYLENPLTAATKAMMSINGDEDSAAALGLLYDYYKVPRDKRLLSVSKASDSQEDQEKRNCLGTSEAQSNLSGGENRVQVLKTVPVNLSLNQDHLENSKREQYSISFPESSAIIPVSGITVVKAEDFTPVFMAPPVHYPRGDGEEQRVVIFEQTQYDVPSLATHSAYLKDDQRSTPDSTYSESFKDAATEKFRSASVGAEEYMYDQTSSGTFQYTLEATKSLRQKQGEGPMTYLNKGQFYAITLSETGDNKCFRHPISKVRSVVMVVFSEDKKQREQLKYWKYWHSRQHTAKQRVLDIADYKESFNTIGNIEEIAYNAVSFTWDVNEEAKIFTITVNCLSTDFSSQKGVKVLPLMIQIDTYSYNNRSNKPIHRAYCQIKVFCDKGAERKIRDEERKQNRKKGKGQASQTQCNSSSDGKLAAIPLQKKSDITFYFKTMPDLHSQPVLFIPDVHFANLQRTCQVYYNTDDEREGGSVLVKRMFRPMEEEFGPVPSKQMKEEGTKRVLLYVRKETDDVFDALMLKSPTVKGLMEAISEKYGLPVEKIAKLYKKSKKGILVNMDDNIIEHYSNEDTFILNMESMVEGFKVTLMEI

SEQ ID NO: 16 - DNA, 21 bp, human
gtactgggtctctctggttag

SEQ ID NO: 17 - DNA, 24 bp, human
cggcggctggctagggatgaagaa

SEQ ID NO: 18 - DNA, 21 bp, human
gagcaagcacaaaccagccaa

SEQ ID NO: 19 - DNA, 11 bp, human
gcccgggccaa

SEQ ID NO: 20 - DNA, 23 bp, human
cacatttctggaaatgcctagat

SEQ ID NO: 21 - DNA, 17 bp, human
agcaggaagaggcgggc

SEQ ID NO: 22 - DNA, 17 bp, human
agcaggaagaggcgggc

SEQ ID NO: 23 - DNA, 15 bp, human
ggggtgagctgggct

SEQ ID NO: 24 - DNA, 20 bp, human
aattctgcctcagtctgcga

SEQ ID NO: 25 - DNA, 17 bp, human
gtctgatttcacaggaa

SEQ ID NO: 26 - DNA, 15 bp, human
ccagtgaggagcggt

SEQ ID NO: 27 - DNA, 25 bp, human
ggcgctggttcccacccagactgtc

SEQ ID NO: 28 - DNA, 20 bp, human
taacatgctctttcttggcc

SEQ ID NO: 29 - DNA, 25 bp, human
ccaacccagaagagctcaggtacat

SEQ ID NO: 30 - DNA, 29 bp, human
gagaatgggcggaactgggcggagttagg

SEQ ID NO: 31 - DNA, 20 bp, human
gctggttctttccgcctcag

SEQ ID NO: 32 - DNA, 29 bp, human
tcgcggcgcgagtttcaggcagcgctgcg

SEQ ID NO: 33 - DNA, 41 bp, human
ggccccgccctctcctcgcggcgcgagtttcaggcagcgct

SEQ ID NO: 34 - DNA, 13 bp, human
ggcgcgagtttca

SEQ ID NO: 35 - DNA, 10 bp, human
cgcgagtttc

SEQ ID NO: 36 - DNA, 21 bp, human
ggcgcgagtttcaggcagcgc